(19) United States
(12) Patent Application Publication
(10) Pub. No.: US 2026/0100250 A1
(43) Pub. Date: Apr. 9, 2026

(54) CUSTOMIZED CODON SEQUENCES

(71) Applicant: NUTCRACKER THERAPEUTICS, INC., Boston, MA (US)

(72) Inventors: Evan Merle MCCARTNEY-MELSTAD, Berkeley, CA (US); Samuel DEUTSCH, Walnut Creek, CA (US)

(21) Appl. No.: 19/418,143

(22) Filed: Dec. 12, 2025

Related U.S. Application Data
(63) Continuation of application No. PCT/US24/33784, filed on Jun. 13, 2024.
(60) Provisional application No. 63/472,647, filed on Jun. 13, 2023.
(60) Provisional application No. 63/656,247, filed on Jun. 5, 2024.

(51) Int. Cl. G16B 30/20

(52) U.S. Cl. CPC G16B 30/20

(57) ABSTRACT

A customized codon sequence may be generated using a method which comprises receiving a target amino acid sequence, generating a plurality of candidate codon sequences, and selecting, from a set of final codon sequences which comprises candidate codon sequences, a customized codon sequence. In such a method, each of the candidate codon sequences may be a codon sequence which codes for the target amino acid sequence, and the final codon sequences may be generated based on a set of initial codon sequences. Additionally, the final codon sequences may be organized into sets of final codon sequences, each of which sets corresponds to a vector from a set of vectors and may comprise codon sequences which are farther from an origin than typical codon sequence from a set of initial codon sequences. Corresponding systems and computer readable mediums for generating customized codon sequences may also be implemented.

### CROSS REFERENCE TO RELATED APPLICATIONS

This application is a continuation of, and claims the benefit of and priority to, International Application No. PCT/US2024/033784, filed Jun. 13, 2024, which claims priority to U.S. Provisional Application Nos. 63/472,647, filed Jun. 13, 2023 and 63/656,247, filed Jun. 5, 2024, the contents of each of which is incorporated by reference herein in their entirety.

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves may also correspond to implementations of the claimed technology.

mRNAs comprise, among other elements, codons (i.e., nucleotide triplets) that code for amino acids, and it is possible that multiple different codons may code for a single amino acid. For example, the amino acid Cysteine could alternatively be coded for by the TGT and TGC codons. As a result, amino acid sequences like the APOE protein can be coded for by many different codon sequences. Indeed, in the case of the APOE protein, there are approximately 6.76*10¹⁶³ potential codon sequences which could be used to code for that exact same protein.

There may be significant differences between codon sequences, even when those codon sequences code for the same amino acid sequences. For example, different codon sequences may have different rates of expression and degradation, or may be more or less difficult to manufacture, even when they code for the same amino acid sequence. However, due to obstacles such as the tremendous size of the design space in which candidate codon sequences corresponding to a given amino acid sequence may be found, existing tools may not be capable of identifying codon sequences which code for desired amino acid sequences while being both manufacturable and able to be efficiently expressed for protein transcription. Accordingly, there is a need in the art for improvements in technology for optimizing codon sequences.

### SUMMARY

Development of methods for optimizing codon sequences may advance the use of polynucleotide-based therapeutic modalities. Design space exploration, either including or followed by selection for manufacturability, may provide advantages in identifying codon sequences which are manufacturable, efficiently expressed and have an increased cellular stability profile. Described herein are devices, systems, and methods for generating candidate codon sequences and selecting a customized sequence from among the candidates. Such methods and systems may be used for improving the manufacture and formulation of biomolecule-containing products, such as therapeutics for individualized care.

An implementation relates to a method comprising receiving a target amino acid sequence; generating a plurality of candidate codon sequences, wherein: each candidate codon sequence codes for the target amino acid sequence; the plurality of candidate codon sequences comprises a set of initial codon sequences and one or more sets of final codon sequences; generating the plurality of candidate codon sequences comprises generating each of the one or more sets of final codon sequences based on the set of initial codon sequences; and for each set of final codon sequences, that set of final codon sequences corresponds to a vector from a set of vectors in a design space; and an average of design space locations of the codon sequences from that set of final codon sequences is farther from an origin than an average of design space locations of the codon sequences from the set of initial codon sequences; selecting, from one of the one or more sets of final codon sequences, a customized codon sequence.

In some implementations of a method such as described in the second paragraph of this summary, generating each of the one or more sets of final codon sequences based on the set of initial codon sequences comprises, for each set of final codon sequences from the one or more sets of final codon sequences: for each step in a sequence of steps: identifying a set of previously generated candidate codon sequences which are farthest from the origin along the along the vector corresponding to that set of final codon sequences; generating a codon weighting table comprising weights based on codon frequencies from the identified set of previously generated candidate codon sequences; and generating a set of new candidate codon sequences based on the codon weighting table; after completing the sequence of steps, selecting candidate codon sequences for that set of final codon sequences.

In some implementations of a method such as described in the third paragraph of this summary, generating the set of new candidate codon sequences based on the codon weighting table comprises, for each of a set of potential candidate codon sequences: performing a set of generation acts comprising: adding a single codon to that potential candidate codon sequence at a probability from the codon weighting table, and at a closest unoccupied location to a first end of that potential candidate codon sequence; and determining if that potential candidate codon sequence satisfies a set of constraints, wherein the set of constraints comprises a set of manufacturability constraints; repeating the set of generation acts until a condition from a set of conditions is satisfied, wherein the set of conditions comprises: that potential candidate codon sequence codes for the target amino acid sequence without violating the set of constraints; and that potential candidate codon sequence is determined to not satisfy the set of constraints.

In some implementations of a method such as described in the fourth paragraph of this summary, for each of the set of potential candidate codon sequences, the first end of that potential candidate codon sequence is a 5′ end of that potential candidate codon sequence.

In some implementations of a method such as described in any of the fourth or fifth paragraphs of this summary, generating the set of new candidate codon sequences based on the codon weighting table comprises: for each of a first subset of the set of potential candidate codon sequences: making a set of positive determinations for that potential candidate codon sequence, wherein the set of positive determinations comprises determining that that potential candidate codon sequence codes for the target amino acid sequence, and determining that that potential candidate codon sequence satisfies the set of constraints; and based on making the set of positive determinations, adding that potential candidate codon sequence to the set of new candidate codon sequences; for each of a second subset of the set of potential candidate codon sequences: determining that that potential candidate codon sequence does not satisfy the set of constraints; and based on determining that that potential candidate codon sequence does not satisfy the set of constraints, adding that potential candidate codon sequence to a failed subsequences table; and the set of constraints comprises not matching any sequences in the failed subsequences table.

In some implementations of a method such as described in any of the fourth through sixth paragraphs of this summary, for each of the set of potential candidate codon sequences, the set of generation acts comprises checking if the closest unoccupied location to the first end of that potential candidate codon sequence corresponds to a fixed codon subsequence; and for at least one of the set of potential candidate codon sequences, at least one repetition of the set of generation acts comprises, based on determining that the closest unoccupied location to the first end of that potential candidate codon sequence corresponds to the fixed codon subsequence, adding the fixed codon subsequence to that potential candidate codon sequence at the closest unoccupied location to the first end of that potential candidate codon sequence.

In some implementations of a method such as described in the second paragraph of this summary, generating each of the one or more sets of final codon sequences based on the set of initial codon sequences comprises, for each set of final codon sequences from the one or more sets of final codon sequences: for each generation in a set of generations: generating a set of new candidate codon sequences by creating a set of mutant codon sequences based on a set of previously generated candidate codon sequences; for each candidate codon sequence in the set of new candidate codon sequences, calculating a fitness score for that candidate codon sequence using a fitness function corresponding to the vector corresponding to that set of final codon sequences; and determining whether a termination condition is satisfied; for each generation in the set of generations other than a final generation, wherein the termination condition is determined to be satisfied in the final generation: identifying a set of candidate codon sequences, based on the identified set of candidate codon sequences not including any candidate codon sequence with a lower fitness score than any candidate codon sequence not comprised by the identified set of candidate codon sequences, as the set of previously generated candidate codon sequences to use for generating the new set of candidate codon sequences in a directly following generation from the set of generations; identifying a set of previously generated candidate codon sequences which are farthest from the origin along the vector corresponding to that set of final codon sequences; and generating a set of new candidate codon sequences by creating a set of mutant codon sequences based on the sequences from the identified set of previously generated candidate codon sequences; and after determining that the termination condition is satisfied, selecting candidate codon sequences for that set of final codon sequences.

In some implementations of a method such as described in the eighth paragraph of this summary, the set of initial codon sequences consists of a single codon sequence which codes for the target amino acid sequence; the one or more sets of final codon sequences consists of a single set of final codon sequences; the set of final codon sequence consists of a single candidate codon sequence; and selecting the customized codon sequence is performed by designating the single candidate codon sequence from the single set of final codon sequences as the customized codon sequence.

In some implementations of a method such as described in any of the second or eighth paragraphs of this summary, for each generation in the set of generations, generating the set of new candidate codon sequences by creating the set of mutant codon sequences based on the set of previously generated candidate codon sequences comprises: identifying a previously generated candidate codon sequence as a parent candidate codon sequence based on the parent candidate codon sequence having a fitness score which is not lower than the fitness score for any other previously generated candidate codon sequence; selecting one or more positions in the parent codon sequence as mutation positions; and defining a child candidate codon sequence by: for each position in the parent codon sequence which is comprised by the mutation positions, defining the child candidate codon sequence as having the same codon in that position as the parent codon sequence; for each position in the parent codon sequence which is comprised by the mutation positions, defining the child candidate codon sequence as having a codon in that position which is synonymous with the codon in that position in the parent codon sequence

In some implementations of a method such as described in the tenth paragraph of this summary, for each generation in the set of generations, selecting one or more positions in the parent codon sequence as mutation positions comprises: at each position from the parent codon sequence, calculating a secondary structure at that position; and selecting the mutation positions based on the calculated secondary structures.

In some implementations of a method such as described in the eleventh paragraph of this summary, selecting mutation positions based on the calculated secondary structures is performed by randomly selecting mutation positions based on the calculated secondary structures.

In some implementations of a method such as described in any of the second through eleventh paragraphs of this summary, selecting the customized codon sequence from one of the one or more sets of final codon sequences comprises: for each codon sequence from the one of the one or more sets of final codon sequences, calculating a self-complementarity score for that final codon sequence by performing acts comprising: generating a set of subsequences for that final codon sequence, wherein each subsequence from the set of subsequences has a length which is equal to the length of each other subsequence from the set of subsequences, and wherein the set of subsequences comprises: each subsequence of that final codon sequence which has the length of each subsequence from the set of subsequences; and each subsequence of a reverse complement of that final codon sequence which has the length of each subsequence from the set of subsequences; for each subsequence from the set of subsequences for that final codon sequence, comparing that subsequence with each other subsequence from the set of subsequences, and creating a set of distance scores comprising one distance score for each of those comparisons; and determining the self-complementarity score by combining the sets of distance scores for each of the subsequences from the set of subsequences; and selecting the customized codon sequence from the one of the one or more sets of final codon sequences based on the self-complementarity scores of the codon sequences from the one of the one or more sets of final codon sequences.

In some implementations of a method as described in the thirteenth paragraph of this summary, for each codon sequence from the one of the one or more sets of final codon sequences: the length of each subsequence from the set of subsequences is 22 nucleotides; and for each comparison between two subsequences from the set of subsequences for that final codon sequence, creating the distance score for that comparison comprises executing instructions operable to: assign distance scores which decrease as the number of differences between the compared subsequences increases, when the number of differences between the compared subsequences is greater than zero and less than a threshold difference level; and assign a minimum distance score when the number of differences between the compared subsequences is greater than the threshold difference level; and selecting the customized codon sequence from the one of the one or more sets of final codon sequences comprises a final codon sequence with a minimum self-complementarity score.

In some implementations of a method as described in any of the second through fourteenth paragraphs of this summary, the design space has at least two dimensions, the at least two dimensions comprising a first dimension and a second dimension, wherein the first dimension and the second dimension are different, and each of the first dimension and the second dimension is selected from: minimum free energy; codon adaptation index; summed frequencies of G and C nucleotides; frequency of U nucleotides; summed or localized probabilities of unpaired bases after folding; modeled or estimated half life; windowed Trifonov linguistic complexity; global Trifonov linguistic complexity; windowed sequence entropy; global sequence entropy; windowed DUST complexity score; global DUST complexity score; and self-complementarity score, wherein, for each candidate codon sequence from the set of candidate codon sequences, a self-complementarity score is calculated for that candidate codon sequence by performing acts comprising: generating a set of subsequences for that codon sequence, wherein each subsequence from the set of subsequences has a length which is equal to the length of each other subsequence from the set of subsequences, and wherein the set of subsequences comprises: each subsequence of that codon sequence which has the length of each subsequence from the set of subsequences; and each subsequence of a reverse complement of that codon sequence which has the length of each subsequence from the set of subsequences; for each subsequence from the set of subsequences for that codon sequence, comparing that subsequence with each other subsequence from the set of subsequences, and creating a set of distance scores comprising one distance score for each of those comparisons; and determining the self-complementarity score by combining the sets of distance scores for each of the subsequences from the set of subsequences.

In some implementations of a method such as described in any of the second through fifteenth paragraphs of this summary, the method comprises: receiving a set of one or more untranslated region sequences; and generating the plurality of candidate codon sequences comprises, for each candidate codon sequence, applying a validation function to that candidate codon sequence by applying the validation function to a nucleotide sequence which comprises that candidate codon sequence.

In some implementations of a method such as described in any of the second through sixteenth paragraphs of this summary, the method comprises generating a seed codon sequence based on providing the target amino acid sequence to a program configured to: generate a plurality of codon sequences which code for the target amino acid sequence; and identify an output codon sequence which has a distance from an origin in a design space corresponding to that program which is greater than an average distance from the origin in the design space corresponding to that program for all of the plurality of codon sequences generated by that program; the seed codon sequence is the output codon sequence identified by the program; and the set of initial codon sequences comprises the seed codon sequence.

In some implementations of a method such as described in the seventeenth paragraph of this summary, the program configured to identify the output codon sequence is configured to generate the plurality of codon sequences which code for the target amino acid in executing a search algorithm.

In some implementations of a method such as described in any of the second through eighteenth paragraphs of this summary, generating each of the one or more sets of final codon sequences based on the set of initial codon sequences comprises, for each set of final codon sequences from the one or more sets of final codon sequences: for each generation in a set of generations: generating a set of new candidate codon sequences based on creating a set of mutant codon sequences based on a set of previously generated candidate codon sequences; and determining whether a termination condition is satisfied.

In some implementations of a method such as described in the nineteenth paragraph of this summary, for each set of final codon sequences from the one or more sets of final codon sequences: for each generation in the set of generations other than an initial generation, the set of previously generated candidate codon sequences is the set of new candidate codon sequence generated on a most recent previous generation; for the initial generation, the set of previously generated candidate codon sequences is the set of initial codon sequences; and for each generation in the set of generations, creating the set of mutant codon sequences based on the set of previously generated candidate codon sequences comprises, for each previously generated candidate codon sequence in the set of previously generated candidate codon sequences: creating a set of unvalidated codon sequences by, for each unvalidated codon sequence from the set of unvalidated codon sequences, mutating a set of codons from that previously generated candidate codon sequence; and generating the set of mutant codon sequences by, for each unvalidated codon sequence from the set of unvalidated codon sequences, applying a validation function to that unvalidated codon sequence.

In some implementations of a method such as described in the twentieth paragraph of this summary, applying the validation function to an unvalidated codon sequence comprises validating manufacturability of the unvalidated codon sequence based on applying a sequence of manufacturability conditions which comprises an initial manufacturability condition and a final manufacturability condition by, for each manufacturability condition in the sequence of manufacturability conditions, performing a set of evaluation tasks comprising: determining whether the unvalidated codon sequence satisfies that manufacturability condition; and in the event that the unvalidated codon sequence does not satisfy that manufacturability condition: mutating a codon in a window corresponding to that manufacturability condition; and repeating the set of evaluation tasks with that manufacturability condition; in the event that the unvalidated codon sequence does satisfy that manufacturability condition: in the event that that manufacturability condition is not the final manufacturability condition, performing the set of evaluation tasks with a next manufacturability condition in the sequence of manufacturability conditions; in the event that that manufacturability condition is the final manufacturability condition and there have been no changes in the unvalidated codon sequence since a most recent performance of the set of evaluation tasks with the initial manufacturability condition, determining the unvalidated codon sequence is a validated output of the validation function; and in the event that that manufacturability condition is the final manufacturability condition and there have been changes in the unvalidated codon sequence since the most recent performance of the set of evaluation tasks with the initial manufacturability condition, performing the set of evaluation tasks with the initial manufacturability condition.

In some implementations of a method such as described in any of the nineteenth through twenty-first paragraphs of this summary, the one or more sets of final codon sequences consists of a single set of final codon sequences; the single set of final codon sequences consists of a single final codon sequence; the set of initial codon sequences comprises a single initial codon sequence; and generating each of the one or more sets of final codon sequences based on the set of initial codon sequences comprises, for each set of final codon sequences from the one or more sets of final codon sequences, for each generation in the set of generations: the set of previously generated candidate codon sequences consists of a single previously generated candidate codon sequence; the set of new candidate codon sequences consists of a single new candidate codon sequence; and generating the set of new candidate codon sequences based on creating the set of mutant codon sequences comprises evaluating each mutant codon sequence from the set of mutant codon sequences with a sequence level fitness function.

In some implementations of a method such as described in the twenty-second paragraph of this summary, generating each of the one or more sets of final codon sequences based on the set of initial codon sequences comprises, for each set of final codon sequences from the one or more sets of final codon sequences, for each generation in the set of generations: determining a mutation count, wherein the mutation count is a number of codons in the single previously generated candidate codon sequence to mutate; and for each mutant codon sequence from the set of mutant codon sequences, determining that mutant codon sequence by performing a set of mutation acts comprising mutating a set of codons from the single previously generated candidate codon sequence, wherein the set of codons has a cardinality equal to the mutation count, and wherein the set of codons mutated for that mutant codon sequence is different from the set of codons mutated for each other mutant codon sequence in that generation.

In some implementations of a method such as described in the twenty-third paragraph of this summary, for each generation in the set of generations, determining the mutation count is performed semi-randomly based on a user input.

In some implementations of a method such as described in any of the twenty-third or twenty-fourth paragraphs of this summary, generating each of the one or more sets of final codon sequences based on the set of initial codon sequences comprises, for each set of final codon sequences from the one or more sets of final codon sequences, for each generation in the set of generations: generating a set of codon fitness scores by, for each codon in the single previously generated candidate codon sequence, calculating a fitness score for that codon using a codon level fitness function; and for each generation in the set of generations, for each mutant codon sequence from the set of mutant codon sequences, mutating the set of codons for that mutant codon sequence comprises randomly mutating individual codons in the single previously generated candidate codon sequence at probabilities based on the fitness scores for those individual codons until a number of codons which has been mutated for that mutant codon sequence is equal to the mutation count.

In some implementations of a method such as described in the twenty fifth paragraph of this summary, for each generation in the set of generations, for each codon in the single previously generated candidate codon sequence, calculating the fitness score for that codon using the codon level fitness function comprises calculating a likelihood of a secondary structure forming at the position of that codon.

In some implementations of a method such as described in the twenty-second paragraph of this summary, for each generation in the set of generations, evaluating each mutant codon sequence from the set of mutant codon sequences with the sequence level fitness function comprises, for each mutant codon sequence from the set of mutant codon sequences, assigning a fitness value to that mutant codon sequence based on a predicted stability for that mutant codon sequence.

In some implementations of a method such as described in the twenty-second paragraph of this summary, for each generation in the set of generations, evaluating each mutant codon sequence from the set of mutant codon sequences with the sequence level fitness function comprises, for each mutant codon sequence from the set of mutant codon sequences: obtaining a set of base degradation values by, for each base from that mutant codon sequence, obtaining a degradation likelihood for that base using a trained machine learning model; and assigning a fitness value to that mutant codon sequence based on the set of base degradation values.

In some implementations of a method such as described in the twenty eighth paragraph of this summary, the trained machine learning model: comprises a set of bidirectional gated recurrent unit layers; has a dropout value of 0.1; and has an output dimensionality of 256 outputs per direction.

In some implementations of a method such as described in any of the twenty second through twenty ninth paragraphs of this summary, for each generation in the set of generations, evaluating each mutant codon sequence from the set of mutant codon sequences with the sequence level fitness function comprises, for each mutant codon sequence from the set of mutant codon sequences obtaining a predicted half life for that mutant codon sequence using a trained machine learning model, wherein: obtaining the predicted half life for that mutant codon sequence using the trained machine learning model comprises: determining a set of features for that mutant codon sequence; and providing the set of features to the trained machine learning model as input; the trained machine learning model comprises: a set of dense layers; and after each layer in the set of dense layers, a dropout layer.

In some implementations of a method such as described in the thirtieth paragraph of this summary, for each mutant codon sequence, the set of features for that mutant codon sequence comprises: codon adaptation index; and a set of features for a 5′ untranslated region of that mutant codon sequence, that mutant codon sequence, and a 3′ untranslated region of that mutant codon sequence, wherein the set of features comprises: minimum free energy; length; guanine-cytosine content; percentage adenine; percentage uracil; percentage guanine; percentage cytosine; QGRS score; RNA binding protein motif count; MicroRNA binding site score; and percentage unpaired bases.

In some implementations of a method such as described in the thirtieth or thirty-first paragraphs of this summary, for each mutant codon sequence, the set of features for that mutant codon sequence comprises a half life provided for that mutant codon sequence provided by a non-deep learning estimator.

In some implementations of a method such as described in any of the thirtieth through thirty-second paragraphs of this summary, for each mutant codon sequence: obtaining the predicted half life for that mutant codon sequence using the trained machine learning model comprises determining a set of per base features for that mutant codon sequence using a separate trained machine learning model, wherein the separate trained machine learning model is trained to provide a degradation likelihood for each base in that mutant codon sequence; the set of per base features comprises: sum of per base degradation means; inverse square root per base degradation means; sum per base activity; inverse square root sum per base reactivity; sum per base reactivity plus degradation means; and inverse square root sum per base reactivity plus degradation means; and the set of features for that mutant codon sequence comprises the set of per base features for that mutant codon sequence.

In some implementations of a method such as described in any of the twenty-second through thirty-third paragraphs of this summary, the method is performed using a computer configured to support a set of threads; and for each generation from the set of generations, the set of mutant codon sequences has a cardinality equal to a cardinality of the set of threads the computer is configured to support.

Another implementation relates to a non-transitory computer readable medium having stored thereon instructions operable to, when executed, cause a processor to perform a method comprising: receiving a target amino acid sequence; generating a plurality of candidate codon sequences, wherein: each candidate codon sequence codes for the target amino acid sequence; the plurality of candidate codon sequences comprises a set of initial codon sequences and one or more sets of final codon sequences; generating the plurality of candidate codon sequences comprises generating each of the one or more sets of final codon sequences based on the set of initial codon sequences; and for each set of final codon sequences, that set of final codon sequences corresponds to a vector from a set of vectors in a design space; and an average of design space locations of the codon sequences from that set of final codon sequences is farther from an origin than an average of design space locations of the codon sequences from the set of initial codon sequences; and selecting, from one of the one or more sets of final codon sequences, a customized codon sequence.

In some implementations of a medium as described in the thirty-fifth paragraph of this summary, generating each of the one or more sets of final codon sequences based on the set of initial codon sequences comprises, for each set of final codon sequences from the one or more sets of final codon sequences: for each generation in a set of generations: generating a set of new candidate codon sequences by creating a set of mutant codon sequences based on a set of previously generated candidate codon sequences; for each candidate codon sequence in the set of new candidate codon sequences, calculating a fitness score for that candidate codon sequence using a fitness function corresponding to the vector corresponding to that set of final codon sequences; and determining whether a termination condition is satisfied; for each generation in the set of generations other than a final generation, wherein the termination condition is determined to be satisfied in the final generation: identifying a set of candidate codon sequences, based on the identified set of candidate codon sequences not including any candidate codon sequence with a lower fitness score than any candidate codon sequence not comprised by the identified set of candidate codon sequences, as the set of previously generated candidate codon sequences to use for generating the new set of candidate codon sequences in a directly following generation from the set of generations; identifying a set of previously generated candidate codon sequences which are farthest from the origin along the vector corresponding to that set of final codon sequences; and generating a set of new candidate codon sequences by creating a set of mutant codon sequences based on the sequences from the identified set of previously generated candidate codon sequences; and after determining that the termination condition is satisfied, selecting candidate codon sequences for that set of final codon sequences.

In some implementations of a medium as described in the thirty-sixth paragraph of this summary, the set of initial codon sequences consists of a single codon sequence which codes for the target amino acid sequence; the one or more sets of final codon sequences consists of a single set of final codon sequences; the set of final codon sequence consists of a single candidate codon sequence; and selecting the customized codon sequence is performed by designating the single candidate codon sequence from the single set of final codon sequences as the customized codon sequence.

In some implementations of a medium as described in the thirty-sixth paragraph of this summary, for each generation in the set of generations, generating the set of new candidate codon sequences by creating the set of mutant codon sequences based on the set of previously generated candidate codon sequences comprises: identifying a previously generated candidate codon sequence as a parent candidate codon sequence based on the parent candidate codon sequence having a fitness score which is not lower than the fitness score for any other previously generated candidate codon sequence; selecting one or more positions in the parent codon sequence as mutation positions; and defining a child candidate codon sequence by: for each position in the parent codon sequence which is comprised by the mutation positions, defining the child candidate codon sequence as having the same codon in that position as the parent codon sequence; for each position in the parent codon sequence which is comprised by the mutation positions, defining the child candidate codon sequence as having a codon in that position which is synonymous with the codon in that position in the parent codon sequence.

In some implementations of a medium as described in the thirty eighth paragraph of this summary, for each generation in the set of generations, selecting one or more positions in the parent codon sequence as mutation positions comprises: at each position from the parent codon sequence, calculating a secondary structure at that position; and selecting the mutation positions based on the calculated secondary structures.

In some implementations of a medium as described in the thirty-ninth paragraph of this summary, selecting mutation positions based on the calculated secondary structures is performed by randomly selecting mutation positions based on the calculated secondary structures.

In some implementations of a medium as described in the thirty-fifth paragraph of this summary, generating each of the one or more sets of final codon sequences based on the set of initial codon sequences comprises, for each set of final codon sequences from the one or more sets of final codon sequences: for each step in a sequence of steps: identifying a set of previously generated candidate codon sequences which are farthest from the origin along the along the vector corresponding to that set of final codon sequences; generating a codon weighting table comprising weights based on codon frequencies from the identified set of previously generated candidate codon sequences; and generating a set of new candidate codon sequences based on the codon weighting table; and after completing the sequence of steps, selecting candidate codon sequences for that set of final codon sequences.

In some implementations of a medium as described in the fortieth paragraph of this summary, generating the set of new candidate codon sequences based on the codon weighting table comprises, for each of a set of potential candidate codon sequences: performing a set of generation acts comprising: adding a single codon to that potential candidate codon sequence at a probability from the codon weighting table, and at a closest unoccupied location to a first end of that potential candidate codon sequence; and determining if that potential candidate codon sequence satisfies a set of constraints, wherein the set of constraints comprises a set of manufacturability constraints; repeating the set of generation acts until a condition from a set of conditions is satisfied, wherein the set of conditions comprises: that potential candidate codon sequence codes for the target amino acid sequence without violating the set of constraints; and that potential candidate codon sequence is determined to not satisfy the set of constraints.

In some implementations of a medium as described in the forty-second paragraph of this summary, for each of the set of potential candidate codon sequences, the first end of that potential candidate codon sequence is a 5′ end of that potential candidate codon sequence.

In some implementations of a medium as described in any of the forty-second or forty-third paragraphs of this summary, generating the set of new candidate codon sequences based on the codon weighting table comprises: for each of a first subset of the set of potential candidate codon sequences: making a set of positive determinations for that potential candidate codon sequence, wherein the set of positive determinations comprises determining that that potential candidate codon sequence codes for the target amino acid sequence, and determining that that potential candidate codon sequence satisfies the set of constraints; and based on making the set of positive determinations, adding that potential candidate codon sequence to the set of new candidate codon sequences; for each of a second subset of the set of potential candidate codon sequences: determining that that potential candidate codon sequence does not satisfy the set of constraints; and based on determining that that potential candidate codon sequence does not satisfy the set of constraints, adding that potential candidate codon sequence to a failed subsequences table; and the set of constraints comprises not matching any sequences in the failed subsequences table.

In some implementations of a medium as described in any of the forty-second through forty-fourth paragraphs of this summary, for each of the set of potential candidate codon sequences, the set of generation acts comprises checking if the closest unoccupied location to the first end of that potential candidate codon sequence corresponds to a fixed codon subsequence; and for at least one of the set of potential candidate codon sequences, at least one repetition of the set of generation acts comprises, based on determining that the closest unoccupied location to the first end of that potential candidate codon sequence corresponds to the fixed codon subsequence, adding the fixed codon subsequence to that potential candidate codon sequence at the closest unoccupied location to the first end of that potential candidate codon sequence.

In some implementations of a medium as described in any of the thirty-fifth through forty-fifth paragraphs of this summary, selecting the customized codon sequence from one of the one or more sets of final codon sequences comprises: for each codon sequence from the one of the one or more sets of final codon sequences, calculating a self-complementarity score for that final codon sequence by performing acts comprising: generating a set of subsequences for that final codon sequence, wherein each subsequence from the set of subsequences has a length which is equal to the length of each other subsequence from the set of subsequences, and wherein the set of subsequences comprises: each subsequence of that final codon sequence which has the length of each subsequence from the set of subsequences; and each subsequence of a reverse complement of that final codon sequence which has the length of each subsequence from the set of subsequences; for each subsequence from the set of subsequences for that final codon sequence, comparing that subsequence with each other subsequence from the set of subsequences, and creating a set of distance scores comprising one distance score for each of those comparisons; and determining the self-complementarity score by combining the sets of distance scores for each of the subsequences from the set of subsequences; and selecting the customized codon sequence from the one of the one or more sets of final codon sequences based on the self-complementarity scores of the codon sequences from the one of the one or more sets of final codon sequences.

In some implementations of a medium as described in the forty sixth paragraph of this summary, for each codon sequence from the one of the one or more sets of final codon sequences: the length of each subsequence from the set of subsequences is 22 codons; and for each comparison between two subsequences from the set of subsequences for that final codon sequence, creating the distance score for that comparison comprises executing instructions operable to: assign distance scores which decrease as the number of differences between the compared subsequences increases, when the number of differences between the compared subsequences is greater than zero and less than a threshold difference level; and assign a minimum distance score when the number of differences between the compared subsequences is greater than the threshold difference level; and selecting the customized codon sequence from the one of the one or more sets of final codon sequences comprises a final codon sequence with a minimum self-complementarity score.

In some implementations of a medium as described in any of the thirty-fifth through forty-seventh paragraphs of this summary, the design space has at least two dimensions, the at least two dimensions comprising a first dimension and a second dimension, wherein the first dimension and the second dimension are different, and each of the first dimension and the second dimension is selected from: minimum free energy; codon adaptation index; summed frequencies of G and C nucleotides; frequency of U nucleotides; windowed Trifonov linguistic complexity; global Trifonov linguistic complexity; windowed sequence entropy; global sequence entropy; windowed DUST complexity score; global DUST complexity score; and self-complementarity score, wherein, for each candidate codon sequence from the set of candidate codon sequences, a self-complementarity score is calculated for that candidate codon sequence by performing acts comprising: generating a set of subsequences for that codon sequence, wherein each subsequence from the set of subsequences has a length which is equal to the length of each other subsequence from the set of subsequences, and wherein the set of subsequences comprises: each subsequence of that codon sequence which has the length of each subsequence from the set of subsequences; and each subsequence of a reverse complement of that codon sequence which has the length of each subsequence from the set of subsequences; for each subsequence from the set of subsequences for that codon sequence, comparing that subsequence with each other subsequence from the set of subsequences, and creating a set of distance scores comprising one distance score for each of those comparisons; and determining the self-complementarity score by combining the sets of distance scores for each of the subsequences from the set of subsequences.

Another implementation relates to a non-transitory computer readable medium having stored thereon instructions for performing a method such as described in any of the sixteenth through thirty-fourth paragraphs of this summary.

Another implementation relates to a machine comprising a network connection and means for generating customized codon sequences.

In some implementations of a machine as described in the fiftieth paragraph of this summary, the means for generating customized codon sequences comprises: means for exploring design space with candidate codon sequences; and means for selecting a customized candidate codon sequence from candidate codon sequences in the design space.

In some implementations of a machine as described in the fifty first paragraph of this summary, the means for exploring design space with candidate codon sequences comprises means for identifying fit candidate codon sequences using a genetic evolutionary algorithm.

Another implementation relates to a method comprising: receiving a target amino acid sequence; receiving a set of one or more untranslated region sequences; and generating a plurality of candidate nucleotide sequences, wherein: each candidate nucleotide sequence comprises a codon sequence that codes for the target amino acid sequence, and each untranslated region sequence from the set of one or more untranslated region sequences; the set of candidate nucleotide sequences comprises an initial nucleotide sequence, one or more intermediate candidate nucleotide sequences, and a final nucleotide sequence; and generating the plurality of candidate nucleotide sequences comprises, for each candidate nucleotide sequence other than the initial nucleotide sequence, performing a set of candidate modification acts on a corresponding previously generated candidate nucleotide sequence.

In some implementations of a method such as described in the fifty-third paragraph of this summary, generating the plurality of candidate nucleotide sequences comprises, for each candidate nucleotide sequence, applying a validation function to an unvalidated candidate nucleotide sequence corresponding to that candidate nucleotide sequence.

In some implementations of a method such as described in the fifty-fourth paragraph of this summary, applying the validation function to an unvalidated nucleotide sequence comprises validating manufacturability of the unvalidated nucleotide sequence based on applying a sequence of manufacturability conditions which comprises an initial manufacturability condition and a final manufacturability condition by, for each manufacturability condition in the sequence of manufacturability conditions, performing a set of evaluation tasks comprising: determining whether the unvalidated codon sequence satisfies that manufacturability condition; and in the event that the unvalidated codon sequence does not satisfy that manufacturability condition: mutating a codon in a window corresponding to that manufacturability condition; and repeating the set of evaluation tasks with that manufacturability condition; in the event that the unvalidated codon sequence does satisfy that manufacturability condition: in the event that that manufacturability condition is not the final manufacturability condition, performing the set of evaluation tasks with a next manufacturability condition in the sequence of manufacturability conditions; in the event that that manufacturability condition is the final manufacturability condition and there have been no changes in the unvalidated codon sequence since a most recent performance of the set of evaluation tasks with the initial manufacturability condition, determining the unvalidated nucleotide sequence is a validated output of the validation function; and in the event that that manufacturability condition is the final manufacturability condition and there have been changes in the unvalidated nucleotide sequence since the most recent performance of the set of evaluation tasks with the initial manufacturability condition, performing the set of evaluation tasks with the initial manufacturability condition.

In some implementations of a method such as described in any of the fifty fourth or fifty fifth paragraphs of this summary, generating the initial nucleotide sequence comprises: providing the target amino acid sequence to a program configured to: generate a plurality of codon sequences which code for the target amino acid sequence; identify an output codon sequence which has a distance from an origin in a design space corresponding to that program which is greater than an average distance from the origin in the design space corresponding to that program for all of the plurality of codon sequences generated by that program; generating a seed nucleotide sequence by combining the output codon sequence identified by the program with the set of one or more untranslated region sequences; and providing the seed nucleotide sequence to the validation function as the unvalidated candidate nucleotide sequence corresponding to the initial nucleotide sequence.

In some implementations of a method such as described in the fifty-sixth paragraph of this summary, the program configured to identify the output codon sequence is configured to generate the plurality of codon sequences which code for the target amino acid sequence in executing a search algorithm.

In some implementations of a method such as described in any of the fifty fourth through fifty seventh paragraphs of this summary, each candidate nucleotide sequence from the plurality candidate nucleotide sequences corresponds to a generation from a set of generations; for each generation in the set of generations other than the generation corresponding to the initial nucleotide sequence: performing the set of candidate modification acts on the corresponding previously generated candidate nucleotide sequence comprises: creating a set of mutant nucleotide sequences based on the corresponding previously generated candidate nucleotide sequence for the candidate nucleotide sequence corresponding to that generation; and generating the candidate nucleotide sequence corresponding to that generation based on the set of mutant nucleotide sequences; and the method comprises determining whether a termination condition is satisfied.

In some implementations of a method such as described in the fifty-eighth paragraph of this summary, for each generation in the set of generations other than the generation corresponding to the initial nucleotide sequence, creating the set of mutant nucleotide sequences comprises: creating a set of unvalidated nucleotide sequences based on, for each unvalidated nucleotide sequence from the set of unvalidated nucleotide sequences, mutating a set of codons from the corresponding previously generated candidate nucleotide sequence for the candidate nucleotide sequence corresponding to that generation; and generating the set of mutant nucleotide sequences by, for each unvalidated nucleotide sequence from the set of unvalidated nucleotide sequences, applying the validation function to that unvalidated nucleotide sequence.

In some implementations of a method such as described in the fifty-ninth paragraph of this summary, for each generation from the set of generations other than the generation corresponding to the initial nucleotide sequence, performing the set of candidate modification acts on the corresponding previously generated candidate nucleotide sequence comprises evaluating each mutant nucleotide sequence from the set of mutant nucleotide sequences with a sequence level fitness function.

In some implementations of a method such as described in the sixtieth paragraph of this summary, for each generation in the set of generations, evaluating each mutant nucleotide sequence from the set of mutant nucleotide sequences with the sequence level fitness function comprises, for each mutant nucleotide sequence from the set of mutant nucleotide sequences, assigning a fitness value to that mutant nucleotide sequence based on a predicted stability for that mutant nucleotide sequence.

In some implementations of a method such as described in the sixtieth paragraph of this summary, for each generation in the set of generations, evaluating each mutant nucleotide sequence from the set of mutant nucleotide sequences with the sequence level fitness function comprises, for each mutant nucleotide sequence from the set of mutant nucleotide sequences: obtaining a set of base degradation values by, for each base from that mutant nucleotide sequence, obtaining a degradation likelihood for that base using a trained machine learning model; and assigning a fitness value to that mutant nucleotide sequence based on the set of base degradation values.

In some implementations of a method such as described in sixty second paragraph of this summary, the trained machine learning model: comprises a set of bidirectional gated recurrent unit layers; has a dropout value of 0.1; and has an output dimensionality of 256 outputs per direction.

In some implementations of a method such as described in the sixtieth paragraph of this summary, for each generation in the set of generations, evaluating each mutant nucleotide sequence from the set of mutant nucleotide sequences with the sequence level fitness function comprises, for each mutant nucleotide sequence from the set of mutant nucleotide sequences obtaining a predicted half life for that mutant nucleotide sequence using a trained machine learning model, wherein: obtaining the predicted half life for that mutant nucleotide sequence using the trained machine learning model comprises: determining a set of features for that mutant nucleotide sequence; and providing the set of features to the trained machine learning model as input; the trained machine learning model comprises: a set of dense layers; and after each layer in the set of dense layers, a dropout layer.

In some implementations of a method such as described in the sixty fourth paragraph of this summary, for each mutant nucleotide sequence, the set of features for that mutant nucleotide sequence comprises: codon adaptation index; and a set of features for a 5′ untranslated region of that mutant nucleotide sequence, a codon sequence comprised by that mutant nucleotide sequence which codes for the target amino acid sequence, and a 3′ untranslated region of that mutant nucleotide sequence, wherein the set of features comprises: minimum free energy; length; guanine-cytosine content; percentage adenine; percentage uracil; percentage guanine; percentage cytosine; QGRS score; RNA binding protein motif count; MicroRNA binding site score; and percentage unpaired bases.

In some implementations of a method such as described in the sixty fourth or sixty fifth paragraphs of this summary, for each mutant nucleotide sequence, the set of features for that mutant nucleotide sequence comprises a half life provided for that mutant nucleotide sequence provided by a non-deep learning estimator.

In some implementations of a method such as described in any of the sixty fourth through sixty sixth paragraphs of this summary, for each mutant nucleotide sequence: obtaining the predicted half life for that mutant nucleotide sequence using the trained machine learning model comprises determining a set of per base features for that mutant nucleotide sequence using a separate trained machine learning model, wherein the separate trained machine learning model is trained to provide a degradation likelihood for each base in that mutant nucleotide sequence; the set of per base features comprises: sum per base degradation means; inverse square root sum per base degradation means; sum per base reactivity; inverse square root sum per base reactivity; sum per base reactivity plus degradation means; and inverse square root sum per base reactivity plus degradation means; and the set of features for that mutant nucleotide sequence comprises the set of per base features for that mutant nucleotide sequence.

In some implementations of a method such as described in any of the fifty ninth through sixty seventh paragraphs of this summary, for each generation in the set of generations, creating the set of unvalidated nucleotide sequences for that generation comprises: determining a mutation count, wherein the mutation count is a number of codons in the corresponding previously generated candidate nucleotide sequence for the candidate nucleotide sequence corresponding to that generation to mutate; and for each unvalidated nucleotide sequence from the set of unvalidated nucleotide sequences for that generation, mutating the set of codons from the corresponding previously generated candidate nucleotide sequence for the candidate nucleotide sequence corresponding to that generation comprises mutating a set of codons from the corresponding previously generated candidate nucleotide sequence, wherein the set of codons has a cardinality equal to the mutation count, and wherein the set of codons mutated for that unvalidated nucleotide sequence is different from the set of codons mutated for each other unvalidated codon sequence in that generation.

In some implementations of a method such as described in the sixty eighth paragraph of this summary, for each generation in the set of generations, determining the mutation count is performed semi-randomly based on a user input.

In some implementations of a method such as described in any of the sixty eighth through sixty ninth paragraphs of this summary, for each generation in the set of generations, other than the generation corresponding to the initial nucleotide sequence: generating the candidate nucleotide sequence corresponding to that generation comprises, generating a set of codon fitness scores by, for each codon in the corresponding previously generated candidate nucleotide sequence for the candidate nucleotide sequence corresponding to that generation, calculating a fitness score for that codon using a codon level fitness function; for each unvalidated nucleotide sequence from the set of unvalidated nucleotide sequences, mutating the set of codons from that unvalidated nucleotide sequences comprises randomly mutating individual codons in the corresponding previously generated candidate nucleotide sequence for the candidate nucleotide sequence corresponding to that generation at probabilities based on the fitness scores for those individual codons until a number of codons which has been mutated for that unvalidated nucleotide sequence is equal to the mutation count.

In some implementations of a method such as described in the seventieth paragraph of this summary, for each generation in the set of generations, for each codon in the corresponding previously generated candidate nucleotide sequence for the candidate codon sequence corresponding to that generation, calculating the fitness score for that codon using the codon level fitness function comprises calculating a likelihood of a secondary structure forming at a position of that codon.

In some implementations of a method such as described in any of the fifty eighth through seventy first paragraphs of this summary, the method is performed using a computer configured to support a set of threads; and for each generation from the set of generations other than the generation corresponding the initial nucleotide sequence, the set of mutant nucleotide sequences has a cardinality equal to a cardinality of the set of threads the computer is configured to support.

Another implementation relates to a non-transitory computer readable medium having stored thereon instructions for performing the method of any of the sixty third through seventy second paragraphs of this summary.

Another implementation relates to a system comprising a computer programmed to perform a method as described in any of the sixty third through seventy second paragraphs of this summary.

It should be appreciated that all combinations of the foregoing concepts and additional concepts discussed in greater detail below (provided such concepts are not mutually inconsistent) are contemplated as being part of the inventive subject matter disclosed herein and to achieve the benefits as described herein.

### BRIEF DESCRIPTION OF THE DRAWINGS

The details of one or more implementations are set forth in the accompanying drawings and the description below. Other features, aspects, and advantages will become apparent from the description, the drawings, and the claims, in which:

FIG. 1 depicts a schematic view of an example of a microfluidic system;

FIG. 2 depicts an exploded perspective view of examples of components of the system of FIG. 1;

FIG. 3 depicts a top plan view of an example of a process chip that may be incorporated into the system of FIG. 1;

FIG. 4 schematically illustrates one variation of an example method of manufacturing an mRNA therapeutic;

FIG. 5 depicts a process which can be used to customize a codon sequence which codes for a given amino acid sequence;

FIG. 6 depicts codon sequences in a two dimensional design space;

FIG. 7 depicts a method which may be used for generating final codon sequences based on initial codon sequences;

FIG. 8 depicts a process for generating codon sequences which code for target amino acid sequences; and

FIG. 9 depicts a process for calculating a self-complementarity score for a codon sequence.

FIG. 10 depicts a method which may be used for generating final codon sequences based on initial codon sequences.

FIG. 11 depicts steps which may be performed in the generation of an initial codon sequence.

FIGS. 12A-12B depict how a manufacturability validation function may be applied.

FIG. 13 depicts how codon sequences may be generated using an evolutionary algorithm.

FIG. 14 depicts a method which may be used in creating an unvalidated sequence from a previously created candidate codon sequence.

FIG. 15 depicts how a sequence could be evaluated using a sequence level fitness function.

FIG. 16 depicts an architecture which could be used for a trained machine learning model functioning as a sequence level fitness function.

FIG. 17 depicts a method for applying a sequence level fitness function based on features for a sequence as a whole.

FIG. 18 depicts a method in which an optimized codon sequence is found using an evolutionary approach where a single candidate nucleotide sequence is generated on each of a plurality of generations based on a candidate nucleotide sequence from a preceding generation.

### DETAILED DESCRIPTION

In some aspects, apparatuses and methods are disclosed herein for customizing codon sequences. In particular, these apparatuses and methods may include design space exploration in which candidate codon sequences are generated, followed by customization in which a customized codon sequence is selected. The apparatuses and methods described herein may be used to obtain codon sequences with enhanced manufacturability, as well as being efficiently expressed and exhibiting enhanced stability.

### Terminology

Throughout this specification and the claims which follow, unless the context requires otherwise, the word “comprise”, and variations such as “comprises” and “comprising” means various components may be co-jointly employed in the methods and articles (e.g., compositions and apparatuses including device and methods). For example, the term “comprising” will be understood to imply the inclusion of any stated elements or steps but not the exclusion of any other elements or steps. In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components, or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is ±0.1% of the stated value (or range of values), ±1% of the stated value (or range of values), ±2% of the stated value (or range of values), ±5% of the stated value (or range of values), ±10% of the stated value (or range of values), etc. Any numerical values given herein should also be understood to include about or approximately that value unless the context indicates otherwise. For example, if the value “10” is disclosed, then “about 10” is also disclosed. Any numerical range recited herein is intended to include all sub-ranges subsumed therein.

It is also understood that when a value is disclosed that “less than or equal to” the value, “greater than or equal to the value,” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although the terms “first” and “second” may be used herein to describe various features/elements (including steps), these features/elements should not be limited by these terms, unless the context indicates otherwise. These terms are used to distinguish one feature/element from another feature/element, and unless specifically pointed out, do not denote a certain order. Thus, a first feature/element discussed below could be termed a second feature/element, and similarly, a second feature/element discussed below could be termed a first feature/element without departing from the teachings of the present invention.

As used herein, “polynucleotide” refers to a nucleic acid molecule containing multiple nucleotides. Aspects of this disclosure include compositions including oligonucleotides having a length of 18-25 nucleotides (e. g., 18-mers, 19-mers, 20-mers, 21-mers, 22-mers, 23-mers, 24-mers, or 25-mers), or medium-length polynucleotides having a length of 26 or more nucleotides (e.g., polynucleotides of 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, or about 300 nucleotides), or long polynucleotides having a length greater than about 300 nucleotides (e.g., polynucleotides of between about 300 to about 400 nucleotides, between about 400 to about 500 nucleotides, between about 500 to about 600 nucleotides, between about 600 to about 700 nucleotides, between about 700 to about 800 nucleotides, between about 800 to about 900 nucleotides, between about 900 to about 1000 nucleotides, between about 300 to about 500 nucleotides, between about 300 to about 600 nucleotides, between about 300 to about 700 nucleotides, between about 300 to about 800 nucleotides, between about 300 to about 900 nucleotides, or about 1000 nucleotides in length, or even greater than about 1000 nucleotides in length. Where a polynucleotide is double-stranded, its length may be similarly described in terms of base pairs.

As used herein “amplification” may refer to polynucleotide amplification. Amplification may include any suitable method for amplification of a polynucleotide and includes, but is not limited to, multiple displacement amplification (MDA), polymerase chain reaction (PCR) amplification, Loop Mediated Isothermal Amplification (LAMP), Nucleic Acid Sequence Based Amplification, Strand Displacement Amplification, Rolling Circle Amplification, and Ligase Chain Reaction.

As used herein a “cassette” (e.g., a synthetic in vitro transcription facilitator cassette) refers to a polynucleotide sequence which may include or be operably linked to one or more expression elements such as an enhancer, a promoter, a leader, an intron, a 5′ untranslated region (UTR), a 3′ UTR, or a transcription termination sequence. In some aspects, a cassette comprises at least a first polynucleotide sequence capable of initiating transcription of an operably linked second polynucleotide sequence (which may comprise a template) and optionally a transcription termination sequence operably linked to the second polynucleotide sequence. The template, as described below, may comprise a sequence of interest, for example, an open reading frame (“ORF”) of interest. The cassette may be provided as a single element or as two or more unlinked elements.

As used herein, a “template” refers to a nucleic acid sequence that contains a sequence of interest for preparing a therapeutic polynucleotide according to the disclosed methods. Templates may be, but are not limited to, a double stranded DNA (dsDNA), an engineered plasmid construct, a cDNA sequence, or a linear nucleic acid sequence (for example, a linear template generated by PCR or by annealing chemically synthesized oligonucleotides). The template may, in certain aspects, be integrated into a “cassette” as described above.

As used herein, the term “sequence of interest” refers to a polynucleotide sequence, the use of which may be deemed desirable for a suitable purpose, in particular, for the manufacture of an mRNA for a therapeutic use, and includes but is not limited to, coding sequences of structural genes, and non-coding regulatory sequences that do not encode and mRNA or protein product.

As used herein, “in vitro transcription” or “IVT” refer to the process whereby transcription occurs in vitro in a non-cellular system to produce synthetic RNA molecules (e.g., synthetic mRNA) for use in various applications, including for therapeutic delivery to a subject, for example, as a therapeutic polynucleotide, which may be part of, or may be used to form, a therapeutic polynucleotide composition as described below. The therapeutic polynucleotide, (e.g., synthetic RNA molecules (transcription product)) generated may be combined with a delivery vehicle to form a therapeutic polynucleotide composition. Synthetic transcription products include mRNAs, antisense RNA molecules, shRNA, circular RNA molecules, ribozymes, and the like. An IVT reaction may use a purified linear DNA template comprising a promoter sequence and the sequence of the open reading frame (ORF) of a sequence of interest, ribonucleotide triphosphates or modified ribonucleotide triphosphates, a buffer system that includes DTT and magnesium ions, and a phage RNA polymerase.

As used herein a “therapeutic polynucleotide” refers to a polynucleotide (e.g., an mRNA) that may be part of a therapeutic polynucleotide composition for delivery to a subject to treat a symptom, disease, or condition in a subject; prevent a symptom, disease, or condition in a subject; or to improve or otherwise modify the subject's health.

As used herein a “therapeutic polynucleotide composition” (or “therapeutic composition” for short) may refer to a composition including one or more therapeutic polynucleotide (e.g., mRNA) encapsulated by a delivery vehicle, which composition may be administered to a subject in need thereof using any suitable administration routes, such as intratumoral, intramuscular, etc. injection. An example of a therapeutic polynucleotide composition is a mRNA nanoparticle comprising at least one mRNA encapsulated by a delivery vehicle molecule (or “delivery vehicle” for short). An mRNA vaccine is one example of a therapeutic polynucleotide composition.

As used herein, “delivery vehicle” refers to any substance that facilitates, at least in part, the in vivo, in vitro, or ex vivo delivery of a polynucleotide (e.g., therapeutic polynucleotide) to targeted cells or tissues (e.g., tumors, etc.). Referring to something as a delivery vehicle need not exclude the possibility of the delivery vehicle also having therapeutic effects. Some versions of a delivery vehicle may provide additional therapeutic effects. In some versions, a delivery vehicle may be a peptoid molecule, such as an amino-lipidated peptoid molecule, that may be used to at least partially encapsulate mRNA.

As used herein, “joining” refers to methods such as ligation, synthesis, primer extension, annealing, recombination, or hybridization use to couple one component to another.

As used herein “purifying” refers to physical and/or chemical separation of a component (e.g., particles) of other unwanted components (e.g., contaminating substances, fragments, etc.).

As used herein, a statement that something is “based on” something else should be understood as meaning that the thing is determined at least in part by what it is identified as being “based on.” When something necessarily is required to be completely determined by something else, it is described as being “based EXCLUSIVELY on” whatever it is completely determined by.

As used herein, “set” means a number, group, or combination of zero or more elements of similar nature, design, or function. It should be understood that a “subset” or a “superset” of a set are not necessarily smaller, or larger, respectively, than the set which they are contained by or which they contain.

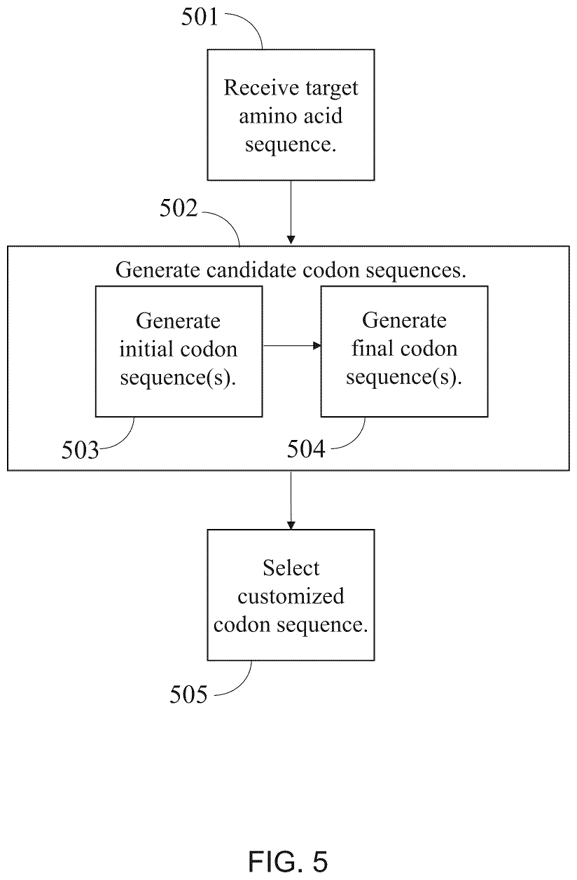

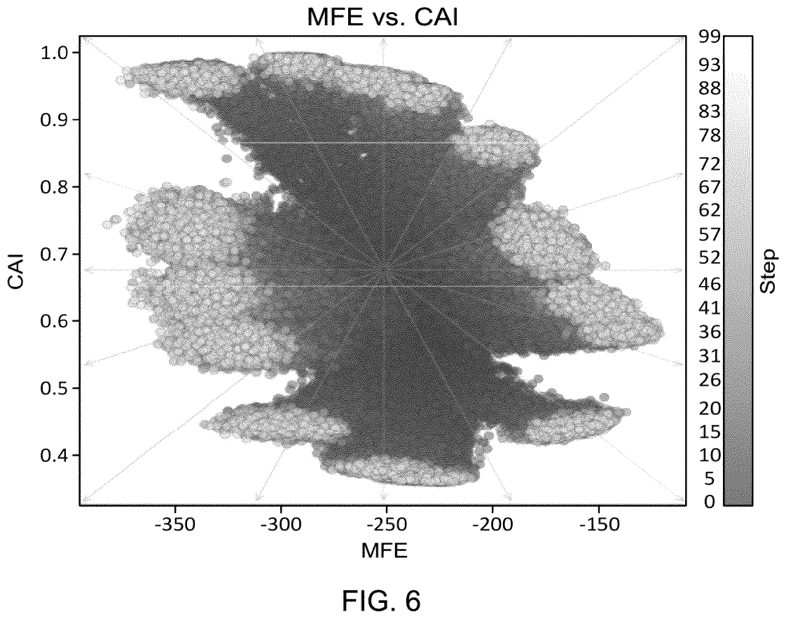

As used herein, “means for generating customized codon sequences” should be understood as a means plus function limitation as provided for in 35 U.S.C. § 112(f), where the function is “generating customized codon sequences” and the corresponding structure is a computer configured to perform processes as illustrated in FIGS. 5, 7-10 and 13 and discussed in the corresponding disclosure of those figures and FIG. 6.

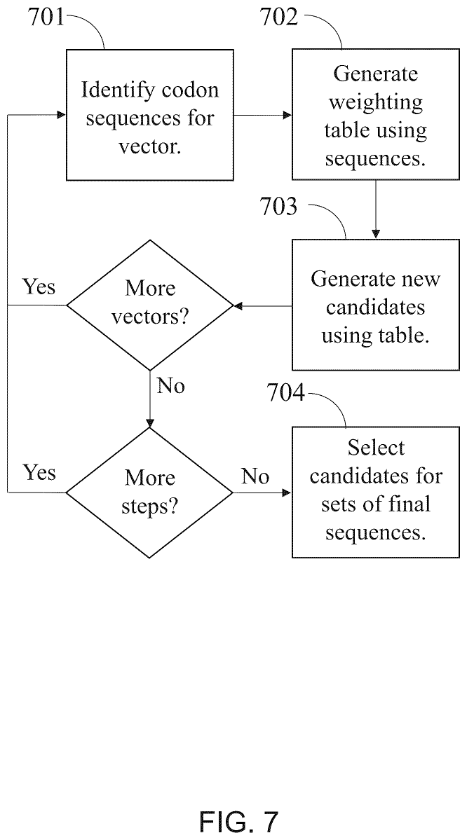

As used herein, “means for exploring design space with candidate codon sequences” should be understood as a means plus function limitation as provided for in 35 U.S.C. § 112(f), where the function is “exploring design space with candidate codon sequences” and the corresponding structure is a computer configured to perform processes as depicted in FIGS. 7-8 and 10 and discussed on the corresponding disclosure of those figures and FIG. 6 (including described variations on potential implementations).

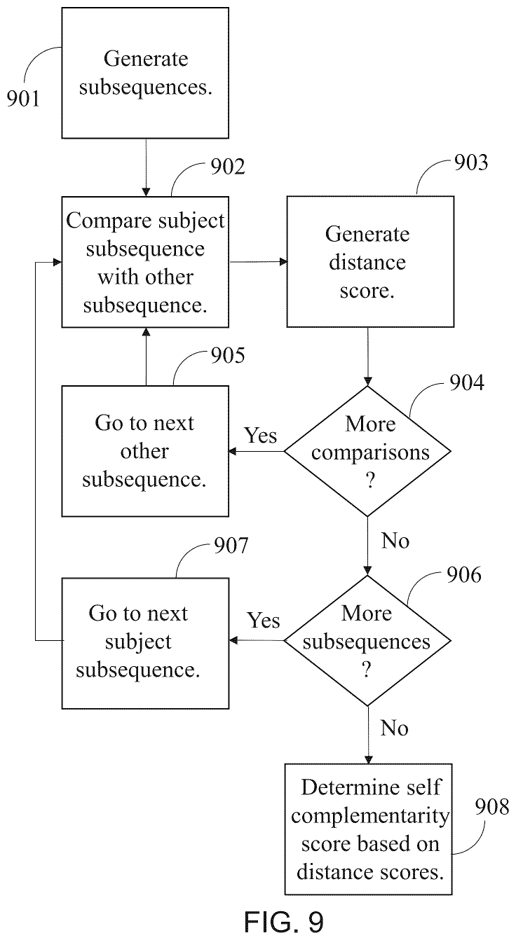

As used herein, “means for selecting a customized candidate codon sequence from candidate codon sequences in the design space” should be understood as a means plus function limitation as provided for in 35 U.S.C. § 112(f), where the function is “selecting a customized candidate codon sequence from candidate codon sequences in design space” and the corresponding structure is a computer configured to perform processes as depicted in FIG. 9 and discussed in the corresponding disclosure (including described variations on potential implementations).

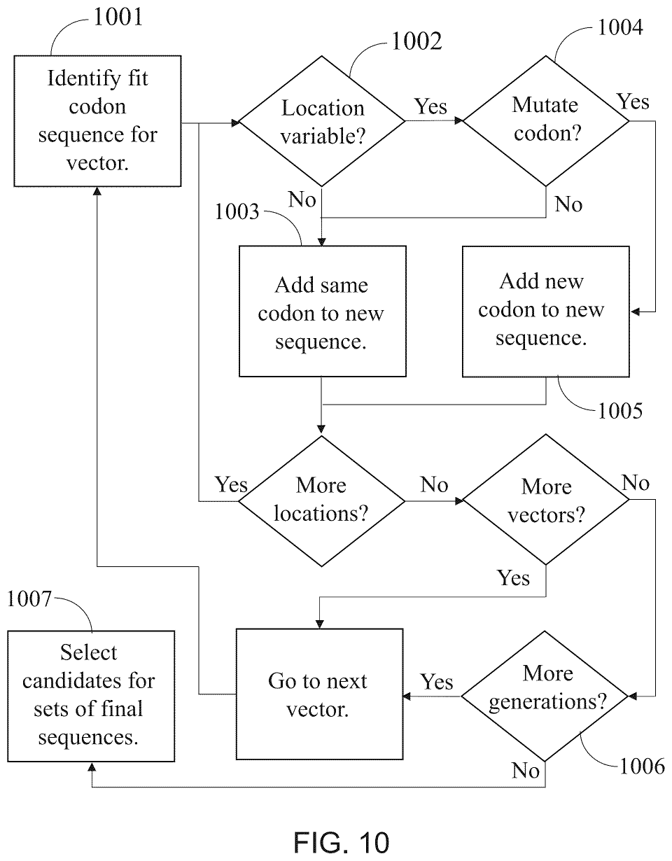

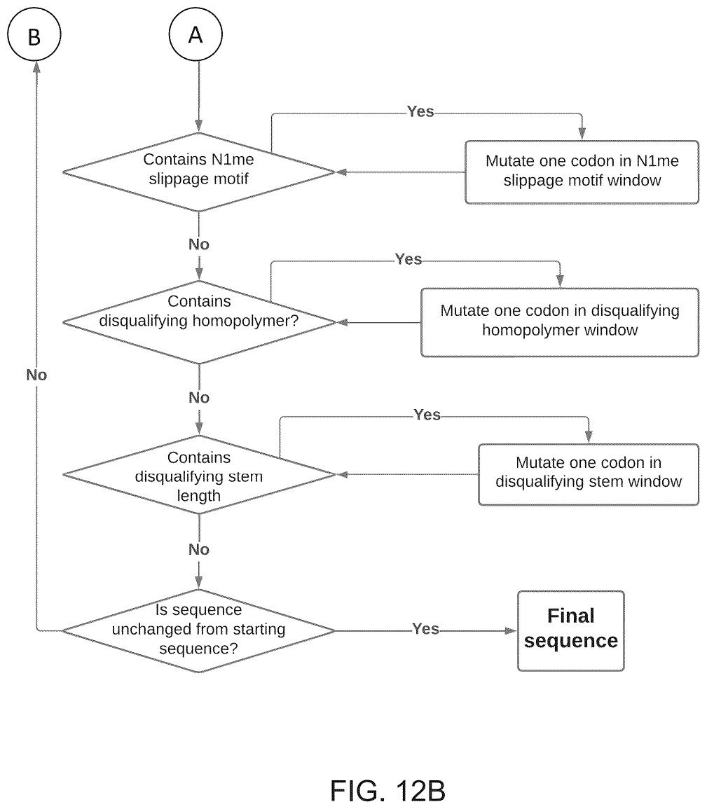

As used herein, “means for identifying fit candidate codon sequences using a genetic evolutionary algorithm” should be understood as a means plus function limitation as provided for in 35 U.S.C. § 112(f), where the function is “identifying fit candidate codon sequences using a genetic evolutionary algorithm” and the corresponding structure is a computer configured to perform processes as depicted in FIGS. 10 and 13 and discussed in the corresponding disclosure (including described variations on potential implementations).

### I. Overview of Synthesis System Including Microfluidic Process Chip

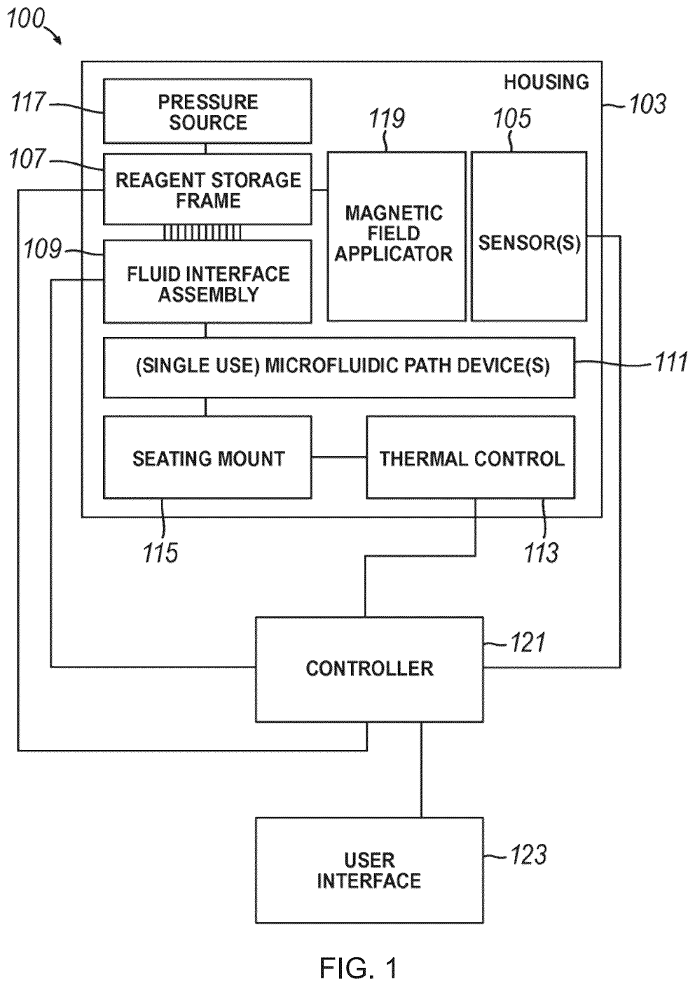

FIG. 1 depicts examples of various components that may be incorporated into a system (100) that could be used for synthesizing codon sequences, such as customized codon sequences created using techniques described herein. System (100) of this example includes a housing (103) enclosing a seating mount (115) that may removably hold one or more microfluidic process chips (111). In other words, system (100) includes a component that is configured to removably accommodate a process chip (111), where the process chip (111) itself defines one or more microfluidic channels or fluid pathways. Components of system (100) (e.g., within housing (103)) that fluidically interact with process chip (111) may include fluid channels or pathways that are not necessarily considered microfluidic (e.g., with such fluid channels or pathways being larger than the microfluidic channels or fluid pathways in process chip (111)). In some versions, process chips (111) are provided and utilized as single-use devices, while the rest of system (100) is reusable. Housing (103) may be in the form of a chamber, enclosure, etc., with an opening that may be closed (e.g., via a lid or door, etc.) to thereby seal the interior. Housing (103) may enclose a thermal regulator and/or may be configured to be enclosed in a thermally-regulated environment (e.g., a refrigeration unit, etc.). Housing (103) may form an aseptic barrier. In some variations, housing (103) may form a humidified or humidity-controlled environment. In addition, or in the alternative, system (100) may be positioned in a cabinet (not shown). Such a cabinet may provide a temperature-regulated (e.g., refrigerated) environment. Such a cabinet may also provide air filtering and air flow management and may promote reagents being kept at a desired temperature through the manufacturing process. In addition, such a cabinet may be equipped with UV lamps for sterilization of process chip (111) and other components of system (100). Other suitable features that may be incorporated into a cabinet that houses system (100). Seating mount (115) may be configured to secure process chip (111) using one or more pins or other components configured to hold process chip (111) in a fixed and predefined orientation. Seating mount (115) may thus facilitate process chip (111) being held at an appropriate position and orientation in relation to other components of system (100). In the present example, seating mount (115) is configured to hold process chip (111) in a horizontal orientation, such that process chip (111) is parallel with the ground.

In some variations, a thermal control (113) may be located adjacent to seating mount (115), to modulate the temperature of any process chip (111) mounted in seating mount (115). Thermal control (113) may include a thermoelectric component (e.g., Peltier device, etc.) and/or one or more heat sinks for controlling the temperature of all or a portion of any process chip (111) mounted in seating mount (115). In some variations, more than one thermal control (113) may be included, such as to separately regulate the temperature of different ones of one or more regions of process chip (111). Thermal control (113) may include one or more thermal sensors (e.g., thermocouples, etc.) that may be used for feedback control of process chip (111) and/or thermal control (113).

As shown in FIG. 1, a fluid interface assembly (109) couples process chip (111) with a pressure source (117), thereby providing one or more paths for fluid (e.g., gas) at a positive or negative pressure to be communicated from pressure source (117) to one or more interior regions of process chip (111) as will be described in greater detail below. While only one pressure source (117) is shown, system (100) may include two or more pressure sources (117). In some scenarios, pressure may be generated by one or more sources other than pressure source (117). For instance, one or more vials or other fluid sources within reagent storage frame (107) may be pressurized. In addition, or in the alternative, reactions and/or other processes carried out on process chip (111) may generate additional fluid pressure. In the present example, fluid interface assembly (109) also couples process chip (111) with a reagent storage frame (107), thereby providing one or more paths for liquid reagents, etc., to be communicated from reagent storage frame (107) to one or more interior regions of process chip (111) as will be described in greater detail below.

In some versions, pressurized fluid (e.g., gas) from at least one pressure source (117) reaches fluid interface assembly (109) via reagent storage frame (107), such that reagent storage frame (107) includes one or more components interposed in the fluid path between pressure source (117) and fluid interface assembly (109). In some versions, one or more pressure sources (117) are directly coupled with fluid interface assembly, such that the positively pressurized fluid (e.g., positively pressurized gas) or negatively pressurized fluid (e.g., suction or other negatively pressurized gas) bypasses reagent storage frame (107) to reach fluid interface assembly (109). Regardless of whether the fluid interface assembly (109) is interposed in the fluid path between pressure source (117) and fluid interface assembly (109), fluid interface assembly (109) may be removably coupled to the rest of system (100), such that at least a portion of fluid interface assembly (109) may be removed for sterilization between uses. As described in greater detail below, pressure source (117) may selectively pressurize one or more chamber regions on process chip (111). In addition, or in the alternative, pressure source may also selectively pressurize one or more vials or other fluid storage containers held by reagent storage frame (107).

Reagent storage frame (107) is configured to contain a plurality of fluid sample holders, each of which may hold a fluid vial that is configured to hold a reagent (e.g., nucleotides, solvent, water, etc.) for delivery to process chip (111). In some versions, one or more fluid vials, or other storage containers in reagent storage frame (107) may be configured to receive a product from the interior of the process chip (111). In addition, or in the alternative, a second process chip (111) may receive a product from the interior of a first process chip (111), such that one or more fluids are transferred from one process chip (111) to another process chip (111). In some such scenarios, the first process chip (111) may perform a first dedicated function (e.g., synthesis, etc.) while the second process chip (111) performs a second dedicated function (e.g., encapsulation, etc.). Reagent storage frame (107) of the present example includes a plurality of pressure lines and/or a manifold configured to divide one or more pressure sources (117) into a plurality of pressure lines that may be applied to process chip (111). Such pressure lines may be independently or collectively (in sub-combinations) controlled.

Fluid interface assembly (109) may include a plurality of fluid lines and/or pressure lines where each such line includes a biased (e.g., spring-loaded) holder or tip that individually and independently drives each fluid and/or pressure line to process chip (111) when process chip (111) is held in seating mount (115). Any associated tubing (e.g., the fluid lines and/or the pressure lines) may be part of fluid interface assembly (109) and/or may connect to fluid interface assembly (109). In some versions, each fluid line comprises a flexible tubing that connects between reagent storage frame (107), via a connector that couples the vial to the tubing in a locking engagement (e.g., ferrule) and process chip (111). In some versions, the ends of the fluid lines/pressure lines, may be configured to seal against process chip (111), e.g., at a corresponding sealing port formed in process chip (111), as described below. In the present example, the connections between pressure source (117) and process chip (111), and the connections between vials in reagent storage frame (107) and process chip (111), all form sealed and closed paths that are isolated when process chip (111) is seated in seating mount (115). Such sealed, closed paths may provide protection against contamination when processing therapeutic polynucleotides.

The vials of reagent storage frame (107) may be pressurized (e.g., >1 atm pressure, such as 2 atm, 3 atm, 5 atm, or higher). In some versions, the vials may be pressurized by pressure source (117). Negative or positive pressure may thus be applied. For example, the fluid vials may be pressurized to between about 1 and about 20 psig (e.g., 5 psig, 10 psig, etc.). Alternatively, a vacuum (e.g., about −7 psig or about 7 psia) may be applied to draw fluids back into the vials (e.g., vials serving as storage depots) at the end of the process. The fluid vials may be driven at lower pressure than the pneumatic valves as described below, which may prevent or reduce leakage. In some variations, the difference in pressure between the fluid and pneumatic valves may be between about 1 psi and about 25 psi (e.g., about 3 psi, about 5 psi, 7 psi, 10 psi, 12 psi, 15 psi, 20 psi, etc.).

System (100) of the present example further includes a magnetic field applicator (119), which is configured to create a magnetic field at a region of the process chip (111). Magnetic field applicator (119) may include a movable head that is operable to move the magnetic field to thereby selectively isolate products that are adhered to magnetic capture beads within vials or other storage containers in reagent storage frame (107).

System (100) of the present example further includes one or more sensors (105). In some versions, such sensors (105) include one or more cameras and/or other kinds of optical sensors. Such sensors (105) may sense one or more of a barcode, a fluid level within a fluid vial held within reagent storage frame (107), fluidic movement within a process chip (111) that is mounted within seating mount (115), and/or other optically detectable conditions. In versions where a sensor (105) is used to sense barcodes, such barcodes may be included on vials of reagent storage frame (107), such that sensor (105) may be used to identify vials in reagent storage frame (107). In some versions, a single sensor (105) is positioned and configured to simultaneously view such barcodes on vials in reagent storage frame (107), fluid levels in vials in reagent storage frame (107), fluidic movement within a process chip (111) that is mounted within seating mount (115), and/or other optically detectable conditions. In some other versions, more than one sensor (105) is used to view such conditions. In some such versions, different sensors (105) may be positioned and configured to separately view corresponding optically detectable conditions, such that a sensor (105) may be dedicated to a particular corresponding optically detectable condition.

In versions where sensors (105) include at least one optical sensor, visual/optical markers may be used to estimate yield. For example, fluorescence may be used to detect process yield or residual material by tagging with fluorophores. In addition, or in the alternative, dynamic light scattering (DLS) may be used to measure particle size distributions within a portion of the process chip (111) (e.g., such as a mixing portion of process chip (111)). In some variations, sensor (105) may provide measurements using one or two optical fibers to convey light (e.g., laser light) into process chip (111); and detect an optical signal coming out of process chip (111). In versions where sensor (105) optically detects process yield or residual material, etc., sensor (105) may be configured to detect visible light, fluorescent light, an ultraviolet (UV) absorbance signal, an infrared (IR) absorbance signal, and/or any other suitable kind of optical feedback.

In versions where sensors (105) include at least one optical sensor that is configured to capture video images, such sensors (105) may record at least some activity on process chip (111). For example, an entire run for synthesizing and/or processing a material (e.g., a therapeutic RNA) may be recorded by one or more video sensors (105), including a video sensor (105) that may visualize process chip (111) (e.g., from above). Processing on process chip (111) may be visually tracked and this video record may be retained for later quality control and/or processing. Thus, the video record of the processing may be saved, stored, and/or transmitted for subsequent review and/or analysis. In addition, as will be described in greater detail below, the video may be used as a real-time feedback input that may affect processing using at least visually observable conditions captured in the video.

System (100) may be controlled by a controller (121). Controller (121) may include one or more processors, one or more memories, and various other suitable electrical components. In some versions, one or more components of controller (121) (e.g., one or more processors, etc.) is/are embedded within system (100) (e.g., contained within housing (103)). In addition, or in the alternative, one or more components of controller (121) (e.g., one or more processors, etc.) may be detachably attached or detachably connected with other components of system (100). Thus, at least a portion of controller (121) may be removable. Moreover, at least a portion of controller (121) may be remote from housing (103) in some versions.

The control by controller (121) may include activating pressure source (117) to apply pressure through process chip (111) to drive fluidic movement, among other tasks. Controller (121) may be completely or partially outside of housing (103); or completely or partially inside of housing (103). Controller (121) may be configured to receive user inputs via a user interface (123) of system (100); and provide outputs to users via user interface (123). In some versions, controller (121) is fully automated to a point where user inputs are not needed. In some such versions, user interface (123) may provide only outputs to users. User interface (123) may include a monitor, a touchscreen, a keyboard, and/or any other suitable features. Controller (121) may coordinate processing, including moving one or more fluid(s) onto and on process chip (111), mixing one or more fluids on process chip (111), adding one or more components to process chip (111), metering fluid in process chip (111), regulating the temperature of process chip (111), applying a magnetic field (e.g., when using magnetic beads), etc. Controller (121) may receive real-time feedback from sensors (105) and execute control algorithms in accordance with such feedback from sensors (105). Such feedback from sensors (105) may include, but need not be limited to, identification of reagents in vials in reagent storage frame (107), detected fluid levels in vials in reagent storage frame (107), detected movement of fluid in process chip (111), fluorescence of fluorophores in fluid in process chip (111), etc. Controller (121) may include software, firmware and/or hardware. Controller (121) may also communicate with a remote server, e.g., to track operation of the apparatus, to re-order materials (e.g., components such as nucleotides, process chips (111), etc.), and/or to download protocols, etc.

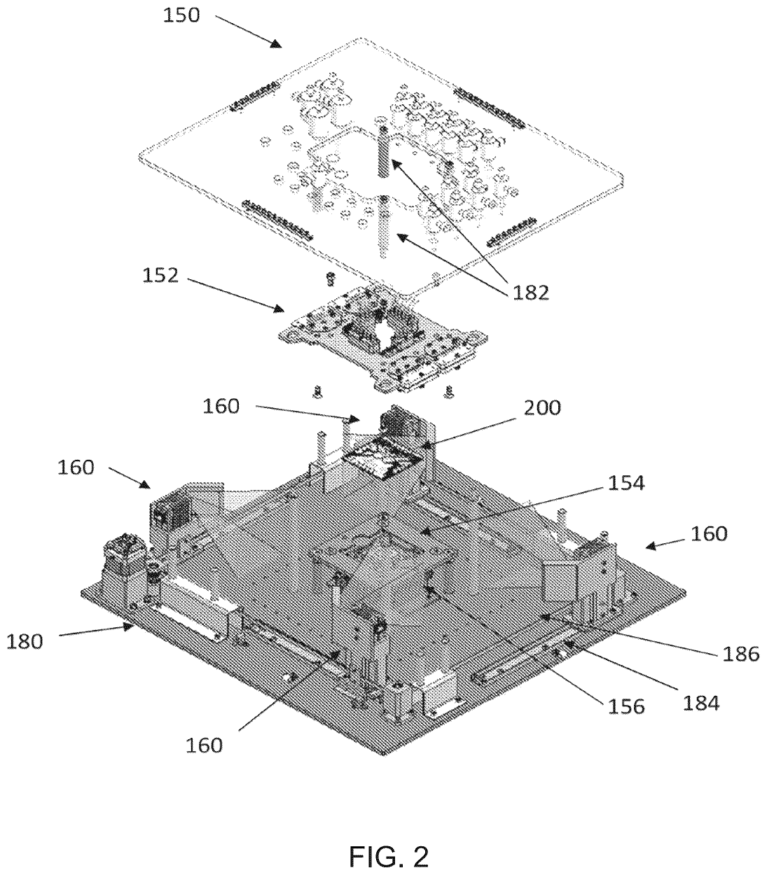

FIG. 2 shows examples of certain forms that may be taken by various components of system (100). In particular, FIG. 2 shows a reagent storage frame (150), a fluid interface assembly (152), a seating mount (154), a thermal control (156), and a process chip (200). Reagent storage frame (150), fluid interface assembly (152), seating mount (154), thermal control (156), and process chip (200) of this example may be configured and operable just like reagent storage frame (107), fluid interface assembly (109), seating mount (115), thermal control (113), and process chip (111), respectively, described above. These components are secured relative to a base (180). A set of rods (182) support reagent storage frame (150) over fluid interface assembly (152).

As shown in FIG. 2, a set of optical sensors (160) are positioned at four respective locations along base (180). Optical sensors (160) may be configured and operable like sensors (105) described above. Optical sensors (160) may include off-the-shelf cameras or any other suitable kinds of optical sensors. Optical sensors (160) are positioned such that fluid vials held within reagent storage frame (150) are within the field of view of one or more of optical sensors (160). In addition, process chip (200) is within the field of view of one or more of optical sensors (160). Each optical sensor (160) is movably secured to base (180) via a corresponding rail (184) (e.g., in a gantry arrangement), such that each optical sensor (160) is configured to translate laterally along each corresponding rail (184). A linear actuator (186) is secured to each optical sensor (160) and is thereby operable to drive lateral translation of each optical sensor (160) along the corresponding rail (184). Each actuator (186) may be in the form of a drive belt, a drive chain, a drive cable, or any other suitable kind of structure. Controller (121) may drive operation of actuators (186). Optical sensors (160) may be moved along rails (184) during operation of system (100) in order to facilitate viewing of the appropriate regions of vials in reagent storage frame (150) and/or process chip (200). In some scenarios, optical sensors (160) move in unison along corresponding rails (184). In some other scenarios, optical sensors (160) move independently along corresponding rails (184).

While optical sensors (160) are shown in FIG. 2 as being mounted to base (180), optical sensors (160) may be positioned elsewhere within system (100), in addition to or as an alternative to being mounted to base (180). For instance, some versions of reagent storage frame (107) may include one or more optical sensors (160) positioned and configured to provide an overhead field of view. In some such versions, such optical sensors (160) may be mounted to rails, movable cantilever arms, or other structures that allow such optical sensors (160) to be repositioned during operation of system (100). Other suitable locations in which optical sensors (160) may be positioned may be used. While not shown, system (100) may also include one or more sources of light (e.g., electroluminescent panels, etc.) to provide illumination that aids in optical sensing by optical sensors (160).

In some versions, one or more mirrors are used to facilitate visualization of components of system (100) by optical sensors (160). Such mirrors may allow optical sensors (160) to view components of system (100) that may not otherwise be within the field of view of sensors (160). Such mirrors may be placed directly adjacent to optical sensors (160). In addition, or in the alternative, such mirrors may be placed adjacent to one or more components of system (100) that are to be viewed by optical sensors (160).

In use of system (100), an operator may select a protocol to run (e.g., from a library of preset protocols), or the user may enter a new protocol (or modify an existing protocol), via user interface (123). From the protocol, controller (121) may instruct the operator which kind of process chip (111) to use, what the contents of vials in reagent storage frame (107) should be, and where to place the vials in reagent storage frame (107). The operator may load process chip (111) into seating mount (115); and load the desired reagent vials and export vials into reagent storage frame (107). System (100) may confirm the presence of the desired peripherals, identify process chip (111), and scan identifiers (e.g., barcodes) for each reagent and product vial in reagent storage frame (107), facilitating the vials to match the bill-of-reagents for the selected protocol. After confirming the starting materials and equipment, controller (121) may execute the protocol. During execution, valves and pumps are actuated to deliver reagents as described in greater detail below, reagents are blended, temperature is controlled, and reactions occur, measurements are made, and products are pumped to destination vials in reagent storage frame (107).

### II. Example of Process Chip

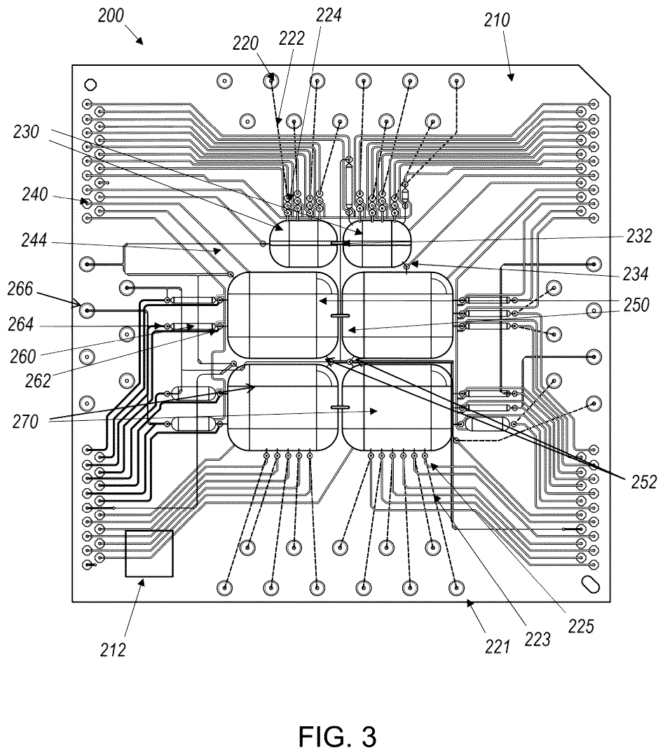

FIG. 3 depicts the example of a process chip (200) in further detail. In combination with the rest of system (100), process chip (200) may be utilized to provide in vitro synthesis, purification, concentration, formulation, and analysis of therapeutic compositions, including but not limited to therapeutic polynucleotides and therapeutic polynucleotide compositions. As shown in FIG. 3, process chip (200) of this example includes a plurality of fluid ports (220). Each fluid port (220) has an associated fluid channel (222) formed in process chip (200), such that fluid communicated into fluid port (220) will flow through the corresponding fluid channel (222). As described in greater detail below, each fluid port (220) is configured to receive fluid from a corresponding fluid line (206) from fluid interface assembly (109). In the present example, each fluid channel (222) leads to a valve chamber (224), which is operable to selectively prevent or permit fluid from the corresponding fluid channel (222) to be further communicated along process chip (200) as will be described in greater detail below.

As also shown in FIG. 3, process chip (200) of this example includes a plurality of additional chambers (230, 250, 270) that may be used to serve different purposes during the process of producing the therapeutic composition. By way of example only, such additional chambers (230, 250, 270) may be used to provide synthesis, purification, dialysis, compounding, and concentration of one or more therapeutic compositions; or to perform any other suitable function(s). Fluid may be communicated from one chamber (230) to another chamber (230) via a fluidic connector (232). In some versions, fluidic connector (232) is operable like a valve between an open and closed state (e.g., similar to valve chamber (224)). In some other versions, fluidic connector (232) remains open throughout the process of making the therapeutic composition. In the present example, chambers (230) are used to provide synthesis of polynucleotides, though chambers (230) may alternatively serve any other suitable purpose(s).

In the example shown in FIG. 3, another valve chamber (234) is interposed between one of chambers (230) and one of chambers (250), such that fluid may be selectively communicated from chamber (230) to chamber (250). Chambers (250) are provided in a pair and are coupled with each other such that process chip (200) may communicate the fluid back and forth between chambers (250). While a pair of chambers (250) are provided in the present example, any other suitable number of chambers (250) may be used, including just one chamber (250) or more than two chambers (250). Chambers (250) may be used to provide purification of the fluid and/or may serve any of the other various purposes described herein; and may have any suitable configuration. In versions where a chamber (250) is used for purification, chamber (250) may include a material that is configured to absorb selected moieties from a fluidic mixture in chamber (250). In some such versions, the material may include a cellulose material, which may selectively absorb double-stranded mRNA from a mixture. In some such versions, the cellulose material may be inserted in only one chamber (250) of a pair of chambers (250), such that upon mixing the fluid from the first chamber (250) of the pair to the second chamber (250), mRNA and/or some other component may be effectively removed from the fluidic mixture, which may then be transferred to another pair of chambers (270) further downstream for further processing or export. Alternatively, chambers (250) may be used for any other suitable purpose.

Additional valve chambers (252) are interposed between each chamber (250) and a corresponding chamber (270), such that fluid may be selectively communicated from chambers (250) to chambers (270) via valve chambers (252). Chambers (270) are also coupled with each other such that process chip (200) may communicate the fluid back and forth between chambers (270). Chambers (270) may be used to provide mixing of the fluid and/or may serve any of the other various purposes described herein; and may have any suitable configuration.

As shown in FIG. 3, chambers (270) are also coupled with additional fluid ports (221) via corresponding fluid channels (223) and valve chambers (225). Fluid ports (221), fluid channels (223), and valve chambers (225) may be configured an operable like fluid ports (220), fluid channels (222), and valve chambers (224) described above. In some versions, fluid ports (221) are used to communicate additional fluids to chambers (270). In addition, or in the alternative, fluid ports (221) may be used to communicate fluid from process chip (200) to another device. For instance, fluid from chambers (270) may be communicated via fluid ports (221) directly to another process chip (200), to one or more vials in reagent storage frame (107), or elsewhere.

Process chip (200) further includes several reservoir chambers (260). In this example, each reservoir chamber (260) is configured to receive and store fluid that is being communicated to or from a corresponding chamber (250, 270). Each reservoir chamber (260) has a corresponding inlet valve chamber (262) and outlet valve chamber (264). Each inlet valve chamber (262) is interposed between reservoir chamber (260) and the corresponding chamber (250, 270) and is thereby operable to permit or prevent the flow of fluid between reservoir chamber (260) and the corresponding chamber (250, 270). Each outlet valve chamber (264) is operable to meter the flow of fluid between reservoir chamber (260) and a corresponding fluid port (266). In some versions, each fluid port (266) is configured to communicate fluid from a corresponding vial in reagent storage frame (107) to a corresponding reservoir chamber (260). In addition, or in the alternative, each fluid port (266) may be configured to communicate fluid from a corresponding reservoir chamber (260) to a corresponding vial in reagent storage frame (107). In the present example, reservoir chambers (260) are used to provide metering of fluid communicated to and/or from process chip (200). Alternatively, reservoir chambers (260) may be utilized for any other suitable purposes, including but not limited to pressurizing fluid that is communicated to and/or from process chip (200).

As also shown in FIG. 3, process chip (200) of this example includes a plurality of pressure ports (240). Each pressure port (240) has an associated pressure channel (244) formed in process chip (200), such that pressurized gas communicated through pressure port (240) will be further communicated through the corresponding pressure channel (244). As described in greater detail below, each pressure port (240) is configured to receive pressurized gas from a corresponding pressure line (208) from fluid interface assembly (109). In the present example, each pressure channel (244) leads to a corresponding chamber (224, 225, 230, 234, 250, 252, 260, 262, 264, 270) to thereby provide valving or peristaltic pumping via such chambers (224, 225, 230, 234, 250, 252, 260, 262, 264, 270) as described in greater detail below.

Process chip (200) may also include electrical contacts, pins, pin sockets, capacitive coils, inductive coils, or other features that are configured to provide electrical communication with other components of system (100). In the example shown in FIG. 3, process chip (200) includes an electrically active region (212) includes such electrical communication features. Electrically active region (212) may further include electrical circuits and other electrical components. In some versions, electrically active region (212) may provide communication of power, data, etc. While electrically active region (212) is shown in one particular location on process chip, electrically active region (212) may alternatively be positioned at any other suitable location or locations. In some versions, electrically active region (212) is omitted.

Some variations of in a process chip (111, 200) may further include a concentration chamber. In some versions of a concentration chamber, polynucleotides may be concentrated by driving off excess fluidic medium, and the concentrated polynucleotide mixture may be exported out of the concentration chamber for further handling or use. In some variations, the concentration chamber may be in the form of a dialysis chamber. For example, a dialysis membrane may be present within or between plates of process chip (111, 200). In some other variations, a concentration chamber may provide concentration without necessarily serving as a dialysis chamber.

The features of process chip (111, 200) described above are non-limiting examples. Additional features that may be incorporated into a process chip (111, 200) are described in greater detail below. Such additional features may be included in a process chip (111, 200) in addition to, or in lieu of, any of the features described above. There may also be scenarios where a plurality of different kinds of process chips (111, 200) are available to serve different kinds of purposes (e.g., to produce different kinds of therapeutic compositions), such that an operator may select the most appropriate process chip on an ad hoc basis to prepare the desired therapeutic substance. Such selections may be made based on the operator's judgment and/or based on the suggestion or instruction from system (100) via user interface (123). In versions where system (100) suggests the kind of process chip (111, 200) to be used, such suggestion may be based on one or more operator inputs provided via user interface (123) and/or based on other factors.

### III. Manufacture of Therapeutics

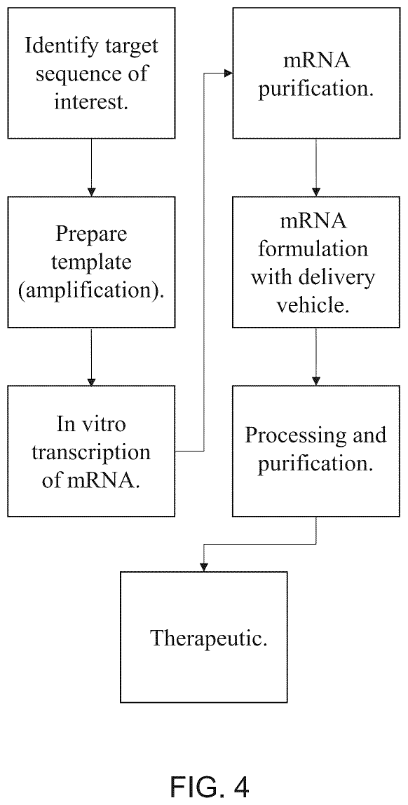

The above-described system may be used for the manufacture of mRNA-based therapeutics. An example of a method for making an mRNA therapeutic is depicted in FIG. 4. In this example method, a target sequence (“sequence of interest”) is identified. A template comprising the target sequence (“sequence of interest”) may then be prepared and amplified (“amplification”) as shown in FIG. 4. Via in vitro transcription of mRNA, mRNA is manufactured using a template comprising the target sequence. The resulting mRNA comprising the sequence of interest may then be purified and formulated with a delivery vehicle. The purification and the formulation may be carried out on the same process chip as the IVT process, on the same process chip only for these two, or on different process chips. The resulting formulation comprising mRNA may then be further processed and optionally purified for a therapeutic use. Such therapeutics uses may include, for example, cell therapies, oncological treatments, protein replacement, vaccines, expression of effector proteins, inducement of loss of function through expression of dominant negative proteins, and gene/genome editing. In addition to their high potency, mRNA therapeutics also have benefits related to their rapid development cycle, standardized manufacturing, transient expression and low risk of genomic integration. The methods and apparatuses described herein may be used to customize codon sequences used in the manufacture mRNA therapeutics for one or more of these categories of therapeutics.

### IV. Codon Sequence Customization

FIG. 5 depicts a process which can be used to customize a codon sequence which codes for a given amino acid sequence, and which could be used for purposes such as manufacture of mRNA therapeutics as described above. As shown, such a process may begin in block 501 with receiving a target amino acid sequence. This may be done, for example, by a user typing a target amino acid sequence into a text entry tool of a computer, and then submitting the sequence to a computer (which may be the user's local computer, if the local computer was configured with codon sequence customization instructions, or may be a remote computer, in the event that codon sequence customization was performed using a cloud platform) which would “receive” it upon the user's submission. As another example, the user may specify a protein (e.g., APOE protein), and the target amino acid sequence may be “received” by execution of a database query retrieving an amino acid sequence corresponding to the protein specified by the user. Other examples, such as receiving a target amino acid sequence through an API, are also possible and will be immediately apparent to those of skill in the art, and so the examples provided of how receiving a target amino acid sequence could be implemented should be understood as being illustrative only, and should not be treated as limiting.

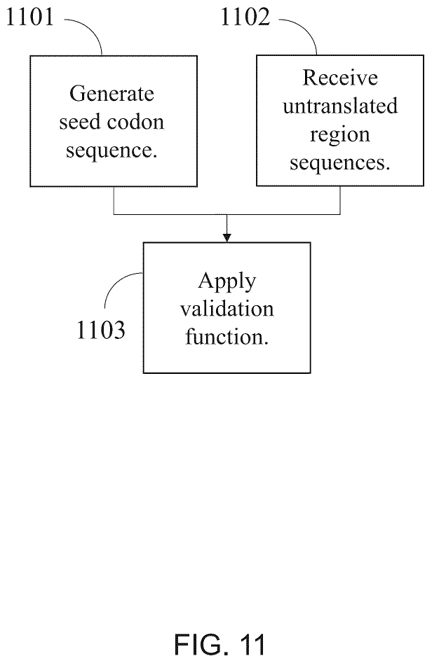

In the process depicted in FIG. 5, after the target amino acid sequence is received in block 501, a plurality of candidate codon sequences is generated in block 502. In this step, each of the candidate codon sequences would code for the target amino acid sequence, and their generation may comprise generating a set of initial codon sequences in block 503, and generating one or more sets of final codon sequences based on the initial codon sequences in block 504. In these sub-steps, the set of initial codon sequences may be one or more candidate codon sequences whose codons are selected randomly from among the codons which code for the amino acids in the target amino acid sequence. However, it is possible that some implementations of the disclosed technology, the generation of initial codon sequences as shown in block 503 may be performed in other manners. An example of this is shown in FIG. 11, discussed below, which illustrates steps which may be performed for the generation of an initial codon sequence in some cases.

As shown in FIG. 11, some implementations of the disclosed technology may include preparatory steps of generating a seed codon sequence in block 1101, and receiving untranslated region sequence(s) in block 1102. Turning to the first of these steps, the generation of a seed codon sequence in block 1101 may be performed by providing the target amino acid sequence to a separate program which is configured to generate a plurality of codon sequences which code for the target amino acid sequence, and then identify an output codon sequence which has a distance from the origin in a design space for that program which is greater than the average distance from the origin of the codon sequences generated by the program. Examples of this type of program include programs implementing the DERNA algorithm described in Xinyu Gu, et. al., DERNA enables Pareto Optimal RNA Design, J Comput Biol. 2024 March; 31 (3): 179-196, doi: 10.1089/cmb.2023.0283. Epub 2024 Feb. 27. PMID: 38416637 and programs implementing the Linear Design algorithm described in Zhang, H., Zhang, L., Lin, A. et al. Algorithm for optimized mRNA design improves stability and immunogenicity. Nature 621, 396-403 (2023), https://doi.org/10.1038/s41586-023-06127-z (each of which is hereby incorporated by reference in its entirety), or other algorithms which executes a search algorithm in its design space for identifying a codon sequence which is optimized according to whatever fitness or evaluation function it uses.

Turning next to the receipt of untranslated regions sequences as shown in block 1102, that act may be done by, for example, receiving a user specification of untranslated regions (e.g., 5′ UTR, 3′UTR) which will ultimately be used when a codon sequence coding for the target amino acid sequence is manufactured. These untranslated region sequences may then be added to a codon sequence which codes for the target amino acid sequence (e.g., a seed codon sequence generated in block 1101), and the combined sequence which both codes for the target amino acid sequence and includes the untranslated regions may have a validation function applied to it in block 1103. This validation function may be, for example, a manufacturability validation function which would test the codon sequence by applying a sequence of manufacturability conditions and adjusting the codon sequence as necessary when a condition was not satisfied. An example of this type of sequence is provided below in table 1, which describes the various conditions as well as the windows where changes may be made in a nucleotide sequence when a condition is found not to be satisfied.

TABLE 1
Illustrative manufacturability conditions

| Condition | Explanation | Window |
|---|---|---|
| Contains | The combined frequency of G and C | The window is the portion of the |
| disqualifying | nucleotides in a sequence having a | sequence in which the |
| % GC windows | particular length, is greater than a | disqualifying GC percentage |
| | threshold allowable percentage. The | was identified. |
| | length of the window and the percentage | |
| | may vary from case to case, and may be | |
| | determined experimentally for the | |
| | instrument which was to manufacture the | |
| | final customized nucleotide sequence. | |
| Contains | There are repeat nucleotide sequences of | The window is the portions of |
| repeated | length k associated with termination of | the nucleotide sequence |
| terminal kmers | transcription within a particular distance | (preferably including the |
| | of each other, which distance may be | untranscribed regions) from the |
| | determined experimentally. | beginning of the first repeated |
| | | terminal kmer to the end of the |
| | | last repeated terminal kmer. |
| Contains bad | There are repeated 2 or 3 nucleotide long | The window is the portion of the |
| 2mer/3mer | sequences (i.e., 2mers or 3mers) which | nucleotide sequence from the |
| repeats | have been found to potentially cause | beginning of the first bad |
| | manufacturing issues within a particular | 2mer/3mer to the end of the last |
| | distance of each other. | 2mer or 3mer. |
| Contains | There is a particular motif in the sequence | The window is the positions in |
| forbidden | which has been identified as forbidden | the nucleotide sequence where |
| motif | (e.g., based on experiments showing that | the motif appears. |
| | RNA which includes that motif is | |
| | particularly difficult to manufacture or | |
| | transcribe). | |
| Contains N1me | A particular motif which has been found | The window is the positions in |
| slippage motif | to interfere with transcription (i.e., the | the nucleotide sequence where |
| | N1me slippage motif, described in | the N1me slippage motif is |
| | Mulroney, T. E., et al., N¹- | found to be present. |
| | methylpseudouridylation of mRNA | |
| | causes + 1 ribosomal | |
| | frameshifting. Nature 625, 189-194 | |
| | (2024). https://doi.org/10.1038/s41586- | |
| | 023-06800-3, which is hereby | |
| | incorporated by reference in its entirety) is | |
| | found to be present. | |
| Contains | The aggregate homopolymer content in | The window is the portion of the |
| disqualifying | either a portion of the sequence having a | sequence where the potentially |
| homopolymer | predefined length or in the sequence as a | problematic homopolymer |
| | whole is found to be greater than a | content is found. |
| | threshold. In this case, the threshold, as | |
| | well as the length of the sequence over | |
| | which that threshold is considered, may be | |
| | determined experimentally by identifying | |
| | characteristics of sequences which are | |
| | found to present particular manufacturing | |
| | difficulty due to homopolymer content. | |
| Contains | A sequence bases which are unlikely to | The window is the sequence of |
| disqualifying | create secondary structure (i.e., the | bases which is unlikely to create |
| stem length | “stem”) is greater than a threshold length | secondary structure. |
| | (the stem length). | |

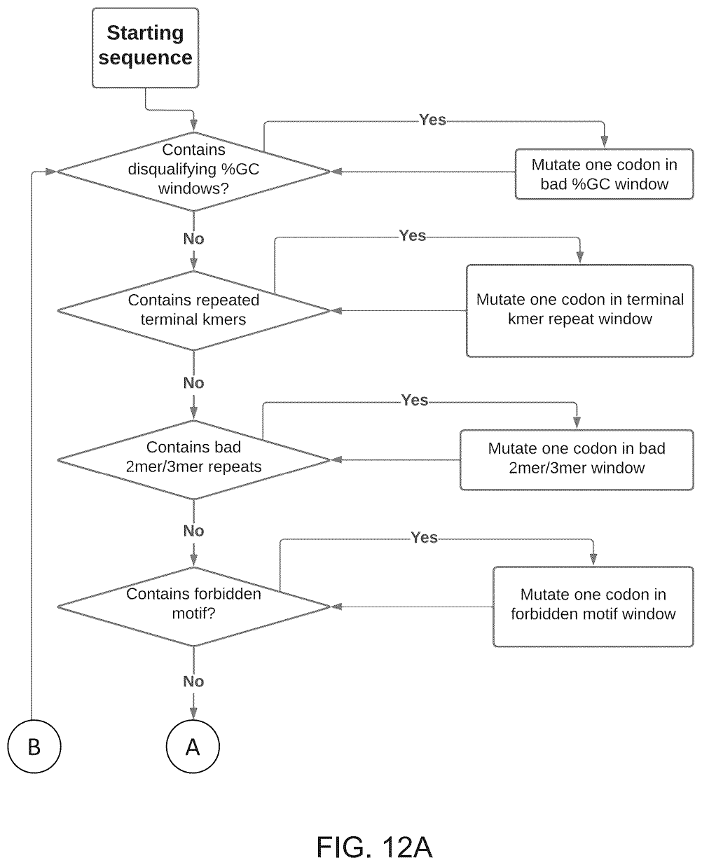

To further illustrate how the application of a validation function from block 1103 may take place, FIGS. 12A-12B illustrate how a manufacturability validation function which applies the conditions of table 1 may be applied. As shown in those figures, the conditions of table 1 may be applied one by one, with each time a condition is found to cause a manufacturability issue, a codon within the window where the condition is discovered can be mutated (i.e., changed with another codon that codes for the same amino acid) to alleviate the condition. This can also cause the condition to be reevaluated for the entire sequence, to address the risk that a change made to fix an issue in one part of a sequence may cause that issue to appear to another part of the sequence instead. Similarly, after all conditions have been evaluated, if a change had been made in the codon sequence to address any of the conditions, the evaluation may return to the first condition in the sequence, to address the risk that a change made to fix an issue identified by one condition may cause an issue corresponding to another condition to appear. Ultimately though, once all conditions had been applied, the final output of the verification function may then be treated as an initial codon sequence for purposes of generating candidate codon sequences such as illustrated in block 502 of FIG. 5.

While FIGS. 12A-12B illustrate how a codon sequence may be validated (including modification as necessary) for manufacturability, it should be understood that those figures, along with the validation criteria of table 1, are intended to be illustrative only, and should not be treated as limiting. For example, in some cases, a validation function may include steps in addition to those illustrated in FIG. 12. For instance, in an implementation which allows a user to specify that certain sequences must be included in a final nucleotide sequence (e.g., untranslated regions, or particular codons that must appear in certain positions), a method such as shown in FIGS. 12A-12B may include steps of determining whether a particular change would violate this type of user specified constraint, and avoid making those changes as part of its manufacturability validation. Similarly, in some cases, a validation function may be configured to address sequences which simply could not be validated (e.g., if a user had specified constraints which would need to be violated for a sequence to be manufacturable). For example, in some cases a validation function may provide a warning message if it was found that a sequence could not be validated and/or may simply not continue with the optimization process, or may continue the optimization process with the sequence that violated the fewest constraints while informing the user of the potential issue(s). Other approaches are also possible, and could be implemented by those of skill in the art without undue experimentation in light of this disclosure, and so the above variations, like the discussion of FIGS. 12A-12B and table 1, should be understood as being illustrative only, and not limiting.

Returning now to the discussion of FIG. 5, however the initial codon sequence(s) are generated (e.g., randomly, using an existing program and validation function, etc.), those initial codon sequences may be used in in block 502's generation of candidate codon sequences by being repeatedly modified until one or more sets of final codon sequences had been generated in block 504. The sets of final codon sequences may be groups of candidate codon sequences which, on average, are at locations in design space which are farther from the origin of the design space (e.g., an intersection point of a set of vectors representing evolutionary fitness functions or other types of evaluation functions, a location representing an average of locations for a set of randomly generated sequences, etc.) than the locations in design space of the initial codon sequences. As an illustration of this, FIG. 6 depicts a plurality of sets of final codon sequences in a two-dimensional design space where the dimensions are the minimum free energy (MFE) and the codon adaptation index (CAI) of the individual codon sequences. In that figure, a set of vectors extends out from an origin point, and, for each vector, there is a corresponding cluster of codon sequences which could be treated as a set of final codon sequences from the one or more sets of final codon sequences generated in block 504. In a design space such as shown in FIG. 6, the origin of the vectors may be defined as a center of the set of initial codon sequences, and the final codon sequences may be generated based on the initial codon sequences using methods such as shown in FIGS. 7, 10 and 13 discussed below.

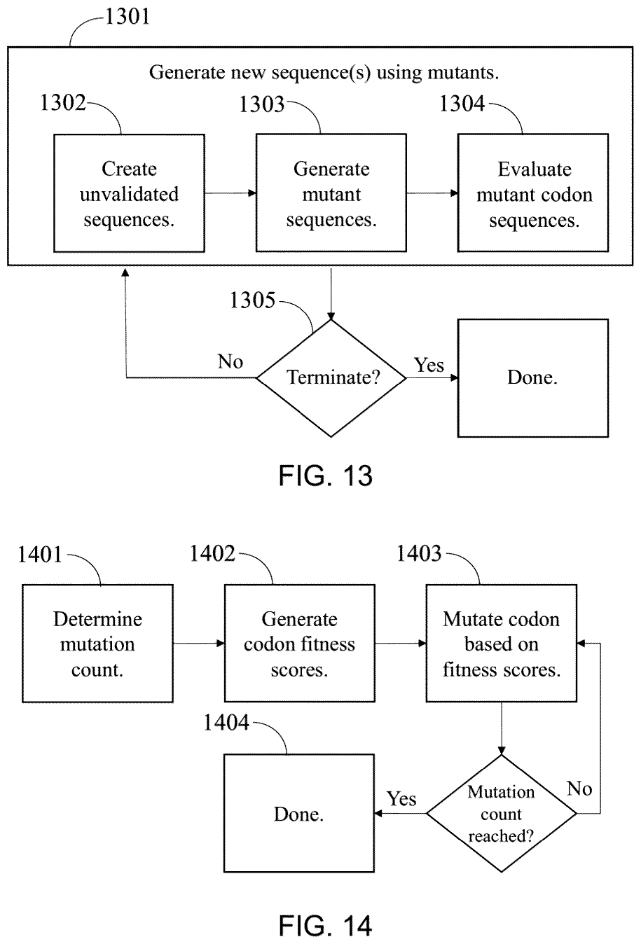

Starting with the method of FIG. 13, as shown in that figure, generating one or more sets of final codon sequences may be performed using an evolutionary algorithm, in each generation, a set of new candidate codon sequences is created in block 1301. This new candidate codon sequence creation may include creating a set of unvalidated codon sequences in block 1302. This unvalidated sequence creation of block 1302 may be performed in a variety of ways. To illustrate, consider FIG. 14, which shows a method which may be used in creating an unvalidated sequence from a previously created candidate codon sequence. Initially, in the method of FIG. 14, a determination is made in block 1401 of how many mutations should be in the unvalidated sequence relative to a progenitor previously created candidate codon sequence. This may be done randomly, such as by randomly selecting a number of codons to mutate, with an upper bound equal to the some portion, such as a one percent, of the number of codons in the progenitor sequence. However, other approaches are also possible. For instance, in some cases, a user may be able to specify a number of codons to mutate, in much the same way he or she might be able to specify step size as a hyperparameter for use in training a deep learning model. Combined approaches are also possible. For example, in some cases the number of mutations may be determined in block 1401 using a semi-random approach in which a user specifies a number of codons to mutate, and the actual number of codons to mutate is determined by drawing from a Poisson distribution where the number specified by the user is the mean number of events. Other approaches (e.g., random or semi-random approaches which use types of distributions other than Poisson distributions, such as gaussian distributions) are also possible, and could be implemented without undue experimentation by one of ordinary skill in light of this disclosure. Accordingly, the above description of how block 1401's determination of a number of codons to mutate should be understood as being illustrative only, and should not be treated as limiting.

Continuing with the discussion of FIG. 14, after the mutation count determination of block 1401, a set of codon fitness scores can be generated in block 1402, such as by using a codon level fitness function to calculate a fitness score for each codon in the progenitor candidate codon sequence. This may include, for each location in the progenitor sequence, calculating a likelihood of a secondary structure forming at the position of that codon. This calculation could be made for a particular codon by calculating the likelihood of each base in that codon pairing with any other base in the sequence which includes the codon (i.e., the progenitor sequence). The codon level fitness function may then treat the combined pairing probability of the codon's bases as its fitness score, or may use that as a parameter for calculating the score. For instance, in some cases, a codon level fitness function may also consider factors such as how each codon contributes to a sequence's codon adaptation index (CAI) score. To illustrate how this may take place, consider a codon which codes for the amino acid phenylalanine and which is part of a sequence in which 10% of the codons which code for phenylalanine are UUU codons, and 90% of the codons which code for phenylalanine are UUC codons. If a CAI table indicated that the optimal codon frequencies for phenylalanine are 75% UUU and 25% UUC a UUC codon may be given a lower fitness score because UUC codons are overrepresented, even if the C base in a UUC codon had a higher likelihood of binding to another base in the sequence. As another example, in cases where a user is allowed to specify constraints for a codon sequence (e.g., that particular codons will appear at particular locations in the sequence), those constraints may also be used in generating the codon level fitness scores (e.g., codons at locations specified by a user may be given a maximum fitness score by default).

However the codon fitness score calculation of block 1402 is performed, once the fitness scores were determined they could be used to mutate one of the progenitor codon sequence's codons in block 1403. This may be done by, for each codon in the parent sequence, mutating that location with a probability determined based on the codon fitness scores determined in block 1402, in which the codons with lower fitness scores (e.g., codons which are less likely to be included in secondary structure) are more likely to be mutated. These mutations could continue until the number of mutations determined in block 1401 had been made, at which point the process could be treated as done in block 1404, and the new sequence could be treated as an unvalidated sequence that could be subjected to further processing in the method of FIG. 13.

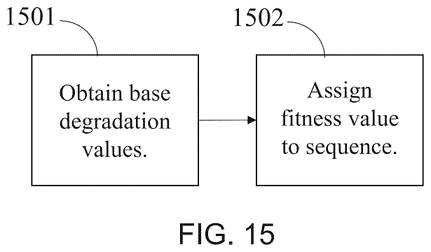

Returning to the process of FIG. 13, once block 1302's creation of unvalidated sequences is complete (e.g., by repeatedly performing the method of FIG. 14, such as by using it to create one unvalidated codon sequence per thread per progenitor codon sequence, so that the unvalidated codon sequences could subsequently be processed in parallel), a set of mutant codon sequences could be generated in block 1303. This may be done by applying a validation function (e.g., a manufacturability validation function such as described previously in the context of table 1 and FIGS. 12A-12B) to each of the unvalidated codon sequences, and treating the result of applying the validation function to the unvalidated codon sequences as the mutant codon sequences. The mutant codon sequences may then be evaluated in block 1304, in order to determine which of those sequences should be treated as new candidate codon sequences for purposes of the evolutionary algorithm. An example of how this type of evaluation could be performed is provided in FIG. 15, discussed below.

Turning now to FIG. 15, that figure illustrates a method for evaluating a sequence by applying a sequence level fitness function. In that method, in block 1501, a set of base degradation values are obtained. To illustrate how this can take place, consider an implementation which uses a trained machine learning model for this purpose. In such an implementation, as a preparatory step for applying the trained machine learning model, a set of features may be extracted so that those features could be used as inputs for the machine learning model. Examples of these types of features include, but are not limited to, those set forth below in table 2.

TABLE 2

| Feature | Explanation |
|---|---|
| One hot coded | A numeric value indicating if the base at the location for which a |
| nucleotide identity | degradation value is being obtained is A, U, C or G. |
| One hot encoded | A numeric value indicating a particular secondary structure in which |
| folded structural | the base is included. Examples of the types of structures which could |
| identity | be represented by this type of one hot coding include stem, dangling |
| | end, hairpin, bulge, multiloop and internal loop. |
| One or more | These summaries can provide information extracted or derived from the |
| summaries of a base | base pairing probability matrix. Examples of such summary features |
| pairing probability | include the sum (i.e., total summed probability that the base is paired |
| matrix (i.e., a matrix | with another base), max (the maximum probability of pairing with |
| showing | another base), non-zeros (how many positions in the sequence have a |
| probabilities that | non-zero probability of binding with this base), over 10 s (how many |
| other bases in the | other positions have at least a 10% chance of binding with this base) |
| sequence will pair | and over 5 s (how many other positions have at least a 5% chance of |
| with the base for | pairing with this base. |
| which a degradation | |
| value is being | |
| obtained). | |
| Global MFE | The minimum free energy for the entire sequence, which may be |
| | repeated for each position in the sequence, if such repetition is needed |
| | given the architecture of the particular machine learning model in |
| | question (e.g., if the machine learning model is a recurrent neural |
| | network). |
| Global GC % | The GC percentage for the entire sequence, which may be repeated if |
| | and as necessary for the particular machine learning model in question. |
| QGRS score | A metric which scores the sequence as a whole for its likelihood to form |
| | g-quadruplexes - a particularly strong type of structural motif. As with |
| | global GC % and Global MFE, this feature may be repeated if and as |
| | necessary for the particular machine learning model in question. |
| One hot encoded | A network graph is constructed from the minimum free energy (folded) |
| graph summarizing | structure of the RNA sequence, where the edges represent covalent or |
| neighboring bases | hydrogen bonds among bases, and nodes represent the bases |
| and structural | themselves. A neighborhood with a radius of 3 is defined for each base, |
| context. | and the combination of distance, base identity, and structural identity is |
| | one-hot encoded for all bases within the neighborhood. For instance, a |
| | position in a folded mRNA molecule may have three positions that are |
| | at a distance of three bonds and that are an A's within stems, one |
| | position at a distance of three bonds that is a U that is part of a bulge, |
| | two positions that are a distance of two bonds that are C's and part of a |
| | multiloop, etc . . . |

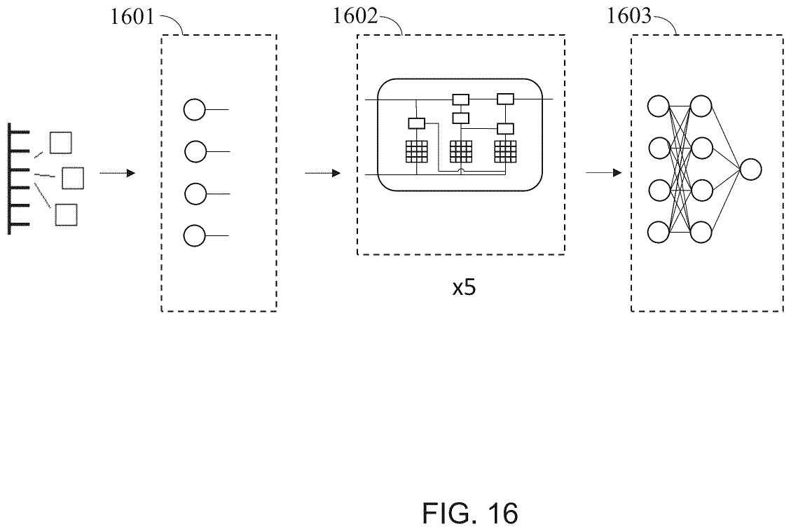

To illustrate how these types of features can be used, consider FIG. 16, which illustrates an architecture for a machine learning model which could be trained to provide degradation values for individual bases in a sequence based on features such as set forth above in table 2. In that architecture, features such as shown in table 2 could be derived for each position in a sequence, and provided to an input layer (1601) which would function as an interface between the machine learning model and the outside world. The input layer (1601) could then pass those features to a series of gated recurrent unit (GRU) layers (1602). In practice, it has been found that a series of five GRU layers, each with a dropout of 0.1, 256 outputs in each directly, a tanh activation function and a sigmoid recurrent activation function are particularly suitable, though other implementations (e.g., using other types of recurrent neural networks, such as long short term memories) are also possible and could be used instead of the 5 GRU layers depicted in FIG. 16. Finally, the output of the last GRU layer is provided as input to a dense layer (1603) with a linear activation function, which dense layer may provide, as its own output, the degradation value for the base whose features were initially provided to the input layer (1601) for processing. Such a machine learning model may be trained to provide base degradation values by using experimentally derived hydrolysis degradation data, such as could be obtained from the Stanford open vaccine challenge (https://www.kaggle.com/c/stanford-covid-vaccine).

Returning now to the method of FIG. 15, once the base degradation values had been obtained in block 1501 (e.g., using a trained machine learning model having an architecture such as shown in FIG. 16), those base degradation values may be used to assign a fitness value to the sequence in block 1502. This may be done, for example, by combining the base degradation values calculated for each of the sequence's bases into a single value which can be used to model the expected half life of the overall sequence, as degradation at any position in the molecule results in degradation of the entire molecule. In implementations which follow this approach, the base degradation values may be combined in a variety of manners, such as by summing them, or by calculating the inverse square root of their sum (i.e., raising the sum of the base degradation values to the −½ power).

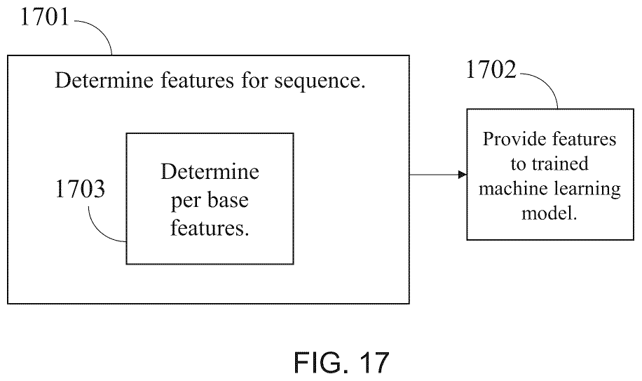

It should be understood that, while the above discussion of table 2 and FIGS. 15-16 provided an example of how a sequence level fitness function could be applied to evaluation of mutant codon sequences, this was intended to be illustrative only, and other evaluation approaches may also be used. As an illustration of such an alternative approach, consider FIG. 17, which depicts an approach in which a sequence level fitness function is applied based on features determined in block 1701 for the sequence as a whole, rather than by determining degradation values for individual bases and then combining those values for the sequence. Examples of features which may be used in this type of evaluation are provided in table 3, below.

TABLE 3

| Feature | Explanation |
|---|---|
| Codon | The codon adaptation index for the entire sequence. |
| adaptation index | |
| (CAI) | |
| Sets of features | A variety of features which are determined for the 5′ UTR, whole sequence, |
| for 5′ UTR, | and 3′ UTR. These can include minimum free energy, length in bases, GC |
| whole sequence, | content, % A, % U, % G, % C, QGRS score, RNA binding protein motif |
| and 3′ UTR | counts (a count of how often sequences that could match one of a predefined |
| | list of RNA binding protein motifs appear in the region in question; the |
| | potential matching sequences can be determined by enumerating all |
| | possible permutations of bases within the position weight matrix that have |
| | a probability greater than some cutoff such as 0.2); MicroRNA binding site |
| | scores (a metric summarizing strength of microRNA binding weighted by |
| | their expression in tissues), and average unpaired percentage. |
| Estimated half | A half life estimate generated for the sequence using a statistical tool such |
| life | as DegScore (developed at Stanford and available at |
| | https://github.com/eternagame/DegScore). |

Once the features have been determined, they may be provided to a trained machine learning model in block 1702, and that model may use the features to provide a predicted half life for the sequence being evaluated. Such a machine learning model may have a variety of architectures. For example, it may begin with an input layer, followed by a set of dense layers (e.g., six dense layers) each of which is followed by a dropout layer (e.g., with a dropout of 0.2), and conclude with a final dense layer having linear activation and one output, which output can be treated as the predicted half life for the sequence from which the features were derived.

It should be understood that, while the method of FIG. 17 was described as illustrating an evaluation on sequence level features as an alternative to the base level features discussed in the context of table 2 and FIGS. 15-16, it is possible that some implementation which use a method such as shown in FIG. 17 may include base level features in the evaluation, as illustrated by the determination of per base features from block 1703. To illustrate how this type of base feature determination may be integrated into the method of FIG. 17, consider a variation on the architecture of FIG. 16 in which the final dense layer (1603) had multiple outputs for generating multiple metrics, rather than simply generating a degradation value as described previously. This type of a variation could be used to evaluate a sequence as part of the determination of per base features, and then the features generated by that evaluation on a per-base level could be consolidated to create sequence level features which could be provided to the input layer of a machine learning model such as described above. For example, an implementation using this approach might utilize a trained machine learning model which would output both a reactivity value and a base degradation value for each base in a sequence. These values could then be used to determine a variety of per base features in block 1703, such as: sum of per-base degradation means, inverse square root sum per base degradation means, sum per-base reactivity, inverse square root sum per-base reactivity, sum per-base reactivity plus degradation means, and inverse square root sum per-base reactivity plus degradation means. Those values could then be treated as features of the overall sequence, and used to generate more accurate half life predictions than may be possible using only metrics such as those set forth above in table 3.

However the evaluation of block 1304 is performed, once it is complete, the evaluation results can be used to determine which codon sequence(s) were suitable for being treated as candidate codon sequences going forward (e.g., the codon sequence with the top score based on the evaluation, the codon sequences with the top N scores based on the evaluation, the top N % of codon sequences based on the evaluation, etc.). A decision can then be made in block 1305 of whether to terminate the candidate codon sequence generation of block 502. If the decision was made to terminate the sequence generation (e.g., because a predefined number of generations had elapsed, because the generation to generation improvement in codon sequences had been below a threshold amount for one or more generations, etc.) then the candidate codon sequence(s) with the highest evaluation values could be treated as the final codon sequence(s). Otherwise, the codon sequences which had been identified as having a sufficiently high evaluation during the preceding generation could be treated as the progenitor candidate codon sequences for a new generation, and the process of FIG. 13 could return to new sequence generation in block 1301 and repeat.

While FIG. 13 illustrated an evolutionary approach to the generation of candidate codon sequences, it is not the only such approach which could be implemented based on this disclosure. As an example of another potential approach, FIG. 10, illustrates another method in which a simulated genetic evolutionary process is used to generate a set of final codon sequences based on a set of initial codon sequences. In the method of FIG. 10, in block 1001, a fit codon sequence will be identified for one of the vectors in the design space. This may be done, for example, by evaluating a previously generated candidate codon sequences using a fitness function corresponding to a vector and treating it as “fit” if it satisfies some criteria when evaluated using that fitness function. For instance, a candidate sequence may be given a fitness score using a function such as fitness score=−aW+bX−cY−dZ, where W is MFE, X is CAI, a and b are coefficients set to match the applicable vector, Y and Z are additional parameters (e.g., percentage uridine; self-complementarity score as determined using a method such as discussed infra in the context of FIG. 9; etc.), and c and d are coefficients of those parameters derived based on empirical data regarding how their respective parameters impact expression, manufacturability or other outcomes in the context in which the disclosed technology is being deployed. This fitness score can then be compared to a cutoff such as a fixed value or a percentile, and, if the fitness score is above the cutoff, then the sequence for which that fitness score was generated can be identified as a “fit” codon sequence.

Once a fit codon sequence had been identified in block 1001, a determination may be made in block 1002 of whether a location in that sequence was variable. This may be done by checking if there was a constraint which would prevent the codon at that location from being changed (e.g., if there was a predefined requirement that the customized codon sequence would have certain codons in certain locations). If there was such a constraint, then, in block 1003, the codon at the evaluated location could simply be added at the same location to a new (child) codon sequence. Otherwise, if the codon could be changed, then a determination could be made in block 1004 of whether it should be changed (mutated). For example, the determination of block 1004 may be made statistically based on a set mutation rate (e.g., 1/1000 chance of a mutation) using a random number, or a pseudo-random number calculated based on secondary structures at the location of the codon which would be changed. If it was determined that the codon should be mutated, then a new codon coding for the same amino acid would be added to the new (child) sequence in block 1005. Otherwise, if there was no mutation, the same codon could be added as described previously in the context of block 1003.

This approach described above for either adding the same codon or a mutated codon could then be repeated for each location in the identified fit sequence, each fit sequence identified for the vector, and each of the vectors defined as corresponding to a fitness function in the design space, thereby creating a new generation of child candidate codon sequences. Once the new generation had been created, a check could be made in block 1006 as to whether that new generation should be the last generation created. This may be done, for example, by checking if a predetermined number of generations had been reached, or if some fitness constraint had been satisfied, such as if the average fitness scores for the most recent generation had exceeded some threshold, or if the fitness score has failed to reach some threshold level of improvement over a predetermined number of generations. If a new generation was needed, then it could be created by re-iterating the mutation and duplication process described above. Otherwise, in block 1007, candidate codon sequences generated during the simulated genetic evolutionary process could be selected for the sets of final sequences. This may be done simply by treating the most recent generation of sequences for each vector as the set of final sequences for that vector. However, other approaches, such as treating the sequences with the highest fitness scores for each vector as the set of final sequences for that vector regardless of those sequences' generations, are also possible.

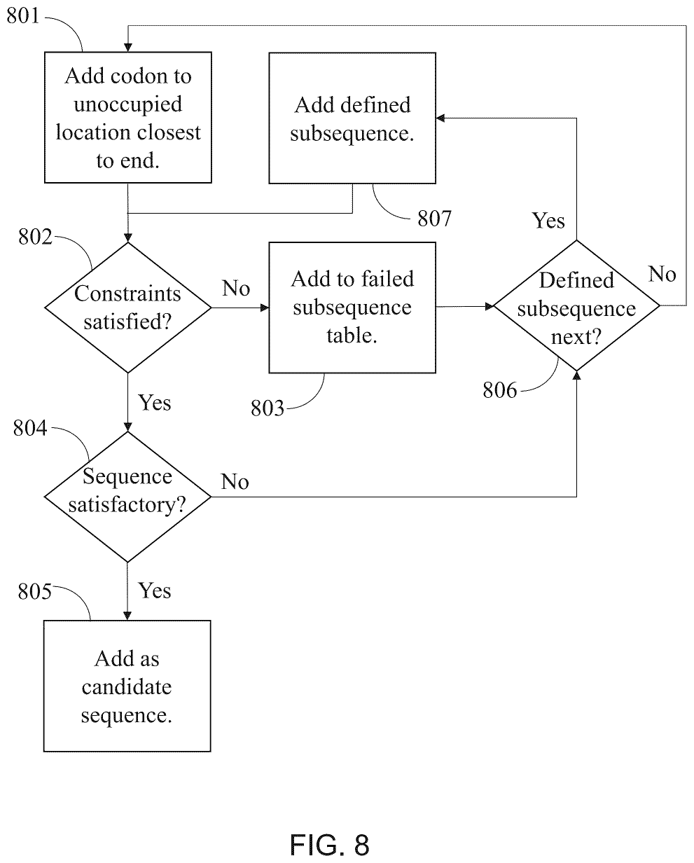

Another approach to generating final codon sequences based on initial codon sequences is shown in FIG. 7. In that figure, in block 701 a set of codon sequences can be identified for one of the vectors in the design space. This may be done by, for example, looking at previously generated candidate codon sequences (e.g., the initial codon sequences) and identifying those sequences which were farthest from the origin along that vector (e.g., the m/2n farthest sequences from the codon sequences in the (n−1)/n percentile of closeness to the vector, where n is the number of vectors and m is the number of codon sequences in the initial codon sequences). The codons in the identified sequences may then be used to generate a new weighting table in block 702, and that new weighting table could be used to generate a new set of candidate codon sequences (e.g., using a process as shown in FIG. 8, discussed infra) in block 703. These acts could then be repeated for each of the vectors under consideration (e.g., the 16 vectors illustrated in FIG. 6), with each time the acts of blocks 701-703 were performed for all vectors being treated as a single step in an iterative design space exploration process. Then, when there were no more steps to be performed (e.g., if blocks 701-703 were performed by a computer program which was programmed to iterate for a fixed number of steps, when that number of steps was reached), the final sets of candidate codon sequences could be selected in block 704, such as by selecting the most recently generated candidate sequences for each vector as the set of final codon sequences for that vector, or by selecting the candidate sequences which were farthest from the origin along each vector as the set of final codon sequences for that vector.

To further illustrate how codon sequences could be identified, FIG. 8 illustrates a method which can be used to generate candidate codon sequences. As shown in FIG. 8, such a method may begin in block 801 with adding a codon to the unoccupied location closest to the end (e.g., the 5′ end) of the codon sequence. This may be done by randomly selecting one of the codons which codes for the next amino acid in the target amino acid sequence with a probability dictated by a weighting table which lists all possible codons along with probabilities that add up to 1. Next, in block 802, a determination may be made as to whether the codon sequence satisfies a set of constraints. These constraints may comprise, for example, manufacturability constraints, which are intended to ensure that the codon sequence can be synthesized on the available hardware (e.g., that it does not include a string of identical nucleotides which is greater than a threshold found to be manufacturable by the machine). Other constraints may also be checked. For example, in some cases, if the sequence being created has already been tried and failed, then it may be added to a failed subsequences table (e.g., as shown in block 803) and the last codon in the sequence may be removed, essentially backtracking to allow an alternative to the failed sequence to be generated. In this type of case, where failed subsequences are added to a failed subsequence table, the constraint check of block 802 may include checking to ensure that the sequence was not included in the failed subsequences table.

Additionally, in some cases a constraint check such as that of block 802 may include checking if the location where the codon was just added should have been occupied by a defined/fixed subsequence. This may be functionality included in embodiments where, rather than simply indicating a target amino acid sequence to be coded for, a user may also specify subsequences of codons which would be required to be used when coding for the target amino acid sequence. In this type of scenario, if a randomly selected codon was added to a location where there should have been a defined/fixed subsequence, the constraint check of block 802 may be deemed to have failed even if the other constraints (e.g., manufacturability) were satisfied. Of course, other types of constraints (e.g., confirmation that the codon sequence under consideration will not coincidentally bind with synthesis primers that would be used to manufacture it in practice, confirmation that the codon sequence under consideration avoids inclusion of a particular restriction enzyme site that may be used in downstream laboratory processes, etc.) are also possible, and will be immediately apparent to those of skill in the art in light of this disclosure. Accordingly, the examples given of determinations which could be included in the constraint check of block 802 should be understood as being illustrative only, and should not be treated as limiting.

Continuing with the discussion of FIG. 8, if the constraints of block 802 were satisfied, then a further check could be made in block 804 of whether the overall sequence was satisfactory (e.g., whether it coded for the target amino acid sequence). If the codon sequence did code for the target amino acid sequence, then all necessary positive determinations (e.g., that it coded for the target amino acid sequence and that it had satisfied the constraints of block 802) could be deemed to have been made. In this case, the codon sequence being generated could be treated as a candidate codon sequence for the purposes of further processing (e.g., further design space exploration using a process such as described above in the context of FIG. 7). Alternatively, if the codon sequence did not code for the target amino acid sequence, further codons could be added until the entire target amino acid sequence was coded for. This may include, for example, checking, in box 806, if the next unoccupied spot in the codon sequence was associated with a defined/fixed subsequence of codons. If so, then the codon sequence could be lengthened by adding the defined/fixed subsequence in block 807. If not, then the codon sequence could be lengthened by adding another codon from the weighting table in block 801 in the manner described previously. The lengthened codon sequence could then be checked for constraints in block 802, checked for whether it was a satisfactory sequence in block 804, and the process could continue until a new candidate codon sequence had been identified.

It should be understood that, while FIG. 8 and the accompanying discussion illustrated how candidate codon sequences could potentially be generated, that figure and its accompanying description are intended to be illustrative only, and that variations could be utilized in some systems or methods implemented based on this disclosure. For example, while the above discussion described initially adding a codon and then checking if it had been added in a location where there should have been a defined/fixed subsequence, in some cases, if a location is allocated for a defined/fixed subsequence, that defined/fixed subsequence may simply be added in that location, rather than initially adding a single codon and then checking if the location where that codon was added was allocated to defined/fixed subsequence. As another example, in some cases, rather than simply populating a failed subsequences table with subsequences that failed constraint checks, a process for identifying candidate codon sequences may check if all possible codons for a location had failed the constraint checks and, if they had, treat the codon sequence which led to that scenario as a failed subsequence. For instance, in an implementation following this approach, if the codon sequence ATG-TGT-CAT needed to be followed by either CAA or CAG in order to continue coding for the target amino acid sequence, if both ATG-TGT-CAT-CAA and ATG-TGT-CAT-CAG failed the constraint checks, then the sequence ATG-TGT-CAT may be treated as failing the constraint check and a new sequence with an alternative to the CAT codon may be evaluated, even if that sequence had passed the constraint check when it was initially considered.

Variations are also possible which diverge entirely from incremental sequence creation such as illustrated in FIG. 8. For example, variations are also possible in the creation of new candidate codon sequences using a simulated evolutionary approach such as shown in FIG. 10. For instance, in some cases, rather than simply mutating fit candidate sequences from a preceding generation, a genetic evolutionary approach may include selecting two fit sequences as parent sequences, then cross them by creating a new sequence whose variable locations are taken from one or the other of its parents (and may also optionally be mutated as well). As another type of variation, different implementations which use a genetic evolutionary approach may use sequences from previous generations as the basis for different numbers of offspring. For example, in some cases, if the initial set of codon sequences has 100 sequences, the top 10 sequences may be considered “fit,” and each of those sequences may be used to create, on average, 10 offspring so that each new generation will have the same size as the initial set of codon sequences. Alternatively, in some cases, only a single codon candidate codon sequence may initially be generated, and each generation may consist of only a single mutated codon sequence per design space vector (if the mutated sequence was more “fit” than the parent with respect to that vector) or of only the parent for that vector (if the parent was more “fit” than the mutated sequence with respect to that vector).

Combinations of the genetic evolutionary approach described in the context of FIG. 10 and the subsequence building approach described in the context of FIG. 7 are also possible. For example, while in some cases the replacement for a codon to be mutated may be selected randomly from all possible codons which would code for the same amino acid, in other cases a codon to be mutated may be replaced with a codon selected with a probability taken from a weighting table in a manner similar to how a weighting table would be used to extend subsequences in the process of FIG. 7. Other variations (e.g., using a different fitness function, or a trained neural network or other machine learning model to evaluate fitness) are also possible, and will be immediately apparent to those of skill in the art in light of this disclosure. Accordingly, the above discussion of variations on the method of FIG. 8, like FIG. 8 itself and its accompanying description, should be understood as being illustrative only, and should not be treated as limiting.

Returning now the discussion of FIG. 5, after the candidate codon sequences had been generated in block 502 (e.g., using processes such as described above in the context of FIGS. 6-8), one of the candidate codon sequences (e.g., a candidate codon sequence from one of the sets of final codon sequences) could be selected as a customized codon sequence in block 505. This may be done, for example, by selecting a set of candidate codon sequences which would be expected to have desirable properties based on their locations in design space, and then identifying one of the codon sequences from that set of candidate codon sequences as the customized codon sequence based on its particular characteristics. For instance, if it was desired to identify a codon sequence which would code for a target amino acid sequence while having a high CAI and low MFE, then the codon sequences from the set of final candidate codon sequences corresponding to the vector pointing to the upper left corner of the design space of FIG. 6 could be checked, and the sequence which was farthest along the specified vector could be selected in block 505 as the customized codon sequence.

Other approaches to selecting a customized codon sequence are also possible, including approaches which incorporate considerations beyond those captured by the dimensions of the design space in which the candidate codon sequences were identified. As an illustration of such an approach, consider an implementation which generates self-complementarity scores (e.g., scores reflecting ease of manufacturability, which may be calculated using a process as depicted in FIG. 9, discussed infra) for selecting a customized codon sequence. In such a case, a set of codon sequences from which the customized codon sequence will be selected will be identified, and a self-complementarity score will be generated for each of those sequences. This set of codon sequences may be selected by, for example, selecting one or more sets of final codon sequences which corresponded to the design space vector that most closely corresponded to the user's desired attributes. For instance, if the disclosed technology was being used by a research lab for generating customized codon sequences to be used for small-scale experiments, then expression may have a relatively high priority, and so the final codon sequences corresponding to vectors with the highest CAI values and lowest MFE values may be used as the codon sequences from which the customized codon sequence will be selected. In some cases, to facilitate this type of customization, a user may be allowed to specify their relative preferences (e.g., manufacturability versus expression) when requesting a customized codon sequence, though other approaches (e.g., having a set of user profiles corresponding to design space weighting, and then allowing the user to select the user profile which most closely matched their objectives) are also possible. In each case though, once the set of potential customized codon sequences had been identified, the actual customized codon sequence could be selected from that set, such as through calculation of self-complementarity scores of each of the potential customized codon sequences.

Turning now to FIG. 9, that figure shows a method of calculating self-complementarity scores for codon sequences. In the method shown in that figure, calculating a self-complementarity score for a codon sequence begins in block 901 with generating subsequences based on that codon sequence. This subsequence generation may comprise identifying every subsequence of the codon sequence which has a defined length (e.g., every 22-nucleotide long subsequence comprised by the nucleotide sequence for which the self-complementarity score is being calculated, every 20-nucleotide long subsequence, every 24-nucleotide long subsequence, etc.), and every subsequence of the reverse complement of the nucleotide sequence which has the defined length. Each of these subsequences may then be compared with each other subsequence on a subsequence-by-subsequence basis. This may begin with comparing one subsequence (referred to for convenience as the “subject” subsequence) with another subsequence in block 902, and generating a distance score based on that comparison in block 903. This may be done by looking at the number of differences between the subject subsequence and the subsequence it was compared with (for instance, by calculating a Hamming distance), and treating the distance score for that comparison as equal to the subsequence length minus the number of differences, or to a different function of the number of differences. For example, in some cases, the distance score for a comparison could be generated using a set of relationships such as that set forth below in table 4.

TABLE 4

| Number of Differences | Distance Score |
|---|---|
| 1 | Assign distance score 10 |
| 2 | Assign distance score 8 |
| 3 | Assign distance score 7 |
| 4 | Assign distance score 6 |
| 5 | Assign distance score 2 |
| Greater than 5 | Assign distance score 0 |

After a distance score had been calculated in block 903, a check may be made in block 904 as to whether there were further comparisons to be made for that subject subsequence—e.g., whether or not there were any other subsequences that that subject subsequence had not yet been compared to. If there were, then the process may proceed to the next subsequence in block 905, and the comparisons may continue until the subject subsequence had been compared with every other subsequence. Alternatively, if that subject subsequence had been compared with every other subsequence, then the process may proceed to determining, in block 906, if there were any more remaining subsequences which had not been compared with at least one other subsequence. If there were any such remaining subsequences, then the process may go to the next subject subsequence in block 907 by designating one of the remaining subsequences which had not been compared with at least one other subsequence as the subject subsequence. The process may then iterate until every subsequence had been compared with every other subsequence, and a distance score had been generated for each of those comparisons. Finally, once all the comparisons were complete, a self-complementarity score for the codon sequence may be determined based on the distance scores generated based on the comparisons of the subsequences in block 908, e.g., by adding up all of the distances scores and treating the sum as the self-complementarity score. Then, once the self-complementarity scores for all of the codon sequences from a set of codon sequences under consideration (e.g., all of the codon sequences from one of the final sets of codon sequences), the codon sequence from that set with the lowest self-complementarity score (i.e., the codon sequence which was likely to be easiest to manufacture) could be selected as the customized codon sequence in block 505 of FIG. 5, and that codon sequence could then be used in practice, such as through the synthesis of physical codon sequences which could be used for purposes such as therapeutics or research as mentioned previously.

It should be understood that, while the above disclosure has provided various examples of how customized codon sequences could be generated, those examples are intended to be illustrative only, and other implementations of the disclosed technology are also possible. For instance, while FIG. 6 illustrated a design space with dimensions of CAI and MFE, other design spaces, such as design spaces which utilize more than two dimensions, and/or use self-complementarity score, GC content (i.e., combined frequency of G and C nucleotides in a sequence), U content (i.e., frequency of U nucleotides in a sequence), aggregate homopolymer content, localized or summed probabilities of unpaired bases after folding, modeled or estimated half life, windowed or global Wootton Federhen sequence complexity (as described in Orlov and Potapov, Complexity: an internet resource for analysis of NDA sequence complexity, published in Nucleic Acids Research, 2004 Jul. 1; 32 (Web Server Issue): W628-W633, the disclosure of which is hereby incorporated by reference in its entirety), windowed or global Trifonov linguistic complexity, windowed or global sequence entropy, or windowed or global DUST complexity scores (as described in Morgulis, et. al., A fast and symmetric DUST implementation to mask low-complexity DNA sequences, published in Journal of Computational Biology, 2006 June; 13(5): 1028-40, the disclosure of which is hereby incorporated by reference in its entirety), or combinations of the foregoing as dimensions, either in addition to, or as alternatives to, MFE and/or CAI, are also possible. As another example, while the discussion of block 505 focused on selecting a customized codon sequence using self-complementarity, it is possible that this selection could be made based on other factors, such as MFE or CAI or one or more of the other potential design space dimensions noted above (e.g., in a case where MFE or CAI or other applicable measure was not used as a design space dimension when generating candidate codon sequences). These factors may also be used as selection criteria (e.g., in the context of checking whether a subsequence satisfies manufacturability constraints in the context of block 802 from FIG. 8), either as alternatives to, or in addition to, those described previously herein.

It is also possible that sets of factors such as those described above could be combined into higher level parameters which be used to help users in properly customizing a codon sequence for their end applications. For example, in some cases factors such as self-complementarity score, GC content, U content, and sequency complexity (e.g., Trifonov complexity scores, DUST complexity scores) could be combined into a single “manufacturability” parameter (e.g., through using a weighted average of the included factors, with the weights being determined based on the impact each of those factors has for manufacturability by the particular hardware which would be used in their synthesis). A user could then be allowed to specify the type of customization he or she desired using an interface which presented a dial, slider or other control allowing “manufacturability” to be balanced against other factors (e.g., a high level “expression” factor based on CAI). This balance could then be used in the generation of candidate codon sequences (e.g., by treating the balanced factors as design space vectors, and selecting the appropriate vector for generating new candidate codon sequences based on the balance specified by the user), in the selection of the customized codon sequence (e.g., by applying the balanced factors to select which of the final codon sequences would be treated as the customized codon sequence), or both.

It is also possible that, in some cases, a system may be implemented based on this disclosure in which one or more measures used as design space dimensions or criteria for selecting a customized codon sequence may change over time. To illustrate, consider a case where a system uses a GC content threshold as a manufacturability constraint to determine whether a subsequence generated in the process of FIG. 8 should be treated as a failed subsequence. In such a case, sensors (e.g., sensors (105) from the system (100) of FIG. 1) may gather information on yield or other quality control parameters and, if the data gathered by those sensors indicated that the then current criteria were inappropriate (e.g., if yields were particularly low) then the GC content threshold (or other applicable constraint) could be adjusted as needed. Similarly, it should be appreciated that all combinations of the foregoing concepts (provided such concepts are not mutually inconsistent) are contemplated as being part of the inventive subject matter disclosed herein and to achieve the benefits as described herein. In particular, all combinations of claimed subject matter appearing at the end of this disclosure are contemplated as being part of the inventive subject matter disclosed herein.

Variations which simplify candidate codon sequence generation and/or customized sequence selection are also possible. To illustrate, consider a case in which a user specifies his or her desired balance of manufacturability and expression before candidate codon sequence generation. In such a case, rather than generating candidate codon sequences by exploring the codon design space along multiple vectors such as shown in FIG. 6, candidate codon sequences may only be generated along a single vector corresponding to the user's desired balance of manufacturability versus expression. Similarly, in some cases, the selection of the customized codon sequence may be performed by simply treating the candidate codon sequence which was the “best” based on the factors used in design space exploration as the customized codon sequence. For instance, in an implementation which uses a genetic evolutionary approach where each generation consists of a single codon sequence, if the user's preference(s) for the customized codon sequence are known, then selection of the customized codon sequence may be performed by simply designating the most “fit” sequence from the genetic evolutionary algorithm as the customized codon sequence.

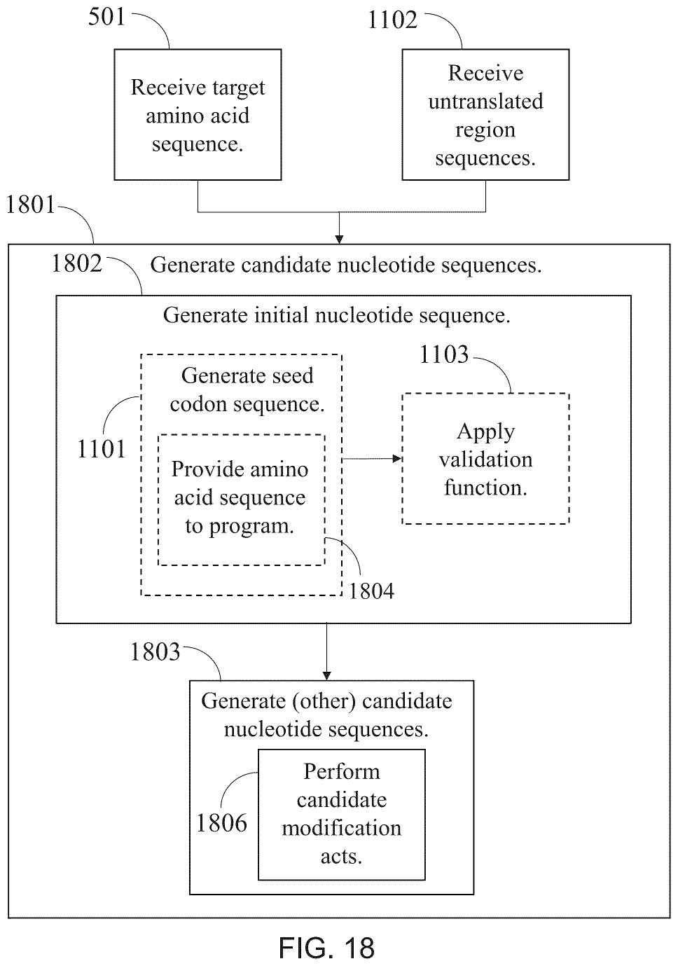

It should be understood that, while the above descriptions provided numerous examples and embodiments, those examples and embodiments are intended to be illustrative only, and the principles and approaches described for one example could be applied in ways beyond those specifically set forth herein. To illustrate, consider that approaches described herein as being performed for multiple sequences, or being performed multiple times for different vectors or fitness functions, may also be applied to individual sequences, or with single vectors or fitness functions. Similarly, a description of an act being performed on a codon sequence should not be understood as implying that that act can only be performed on a sequence made up purely of codons, but instead should be understood as indicating that that act could be performed for a sequence which comprises codons, but which may also include other nucleotides as well (e.g., untranslated regions). A diagram depicting this is provided in FIG. 18, discussed below.

Turning now to FIG. 18, that figure illustrates a method in which an optimized codon sequence is found using an evolutionary approach where a single candidate nucleotide sequence is generated on each of a plurality of generations based on a candidate nucleotide sequence from a preceding generation. In that method, initially, target amino acid sequences and untranslated region sequences may be received in blocks 501 and 1102, with those block numbers being reused to indicate that these acts may be performed in the manner previously described in the context of FIGS. 5 and 11. The method may continue in block 1801 with generating candidate nucleotide sequences, which may be considered as a special case of generating candidate codon sequences as described for block 502 of FIG. 5. As shown in FIG. 18, in a case where there is only one nucleotide sequence per generation, generating candidate nucleotide sequences may include generating an initial nucleotide sequence in block 1802 and generating other candidate nucleotide sequences (e.g., intermediate candidate nucleotide sequences and a final nucleotide sequence) in block 1803, with the generation of blocks 1802 and 1803 being performed using acts described previously despite the fact that the method of FIG. 18 depicts a method which could be performed in a single candidate per generation implementation. Specifically, the initial nucleotide sequence could be generated by generating a seed codon sequence in block 1101 by providing the target amino acid sequence to a program which is configured to search its respective design space for an optimized codon sequence in block 1804, and then applying a validation function to a seed nucleotide sequence made up of the seed codon sequence and the untranslated region sequences in block 1103. The remaining candidate nucleotide sequences could then be generated in block 1803 by performing a set of candidate modification acts in block 1806. These candidate modification acts could take a candidate codon for each generation, and use that candidate codon as a basis for creating a new candidate codon for the next generation, such as through creating mutant codon sequences using a codon level fitness function and a validation function as described in the context of FIGS. 13 and 14, then identifying a single one of those mutant codon sequences to be the candidate codon sequence for the next generation using a sequence level fitness function as described in the context of FIGS. 15-17. This could continue until some termination condition was reached (e.g., as described in the context of block 1305 from FIG. 13), at which point the most recently created candidate nucleotide sequence could be identified as the final codon sequence, and therefore an optimized codon sequence for the target amino acid sequence and specified untranscribed regions.

Other modifications and variations beyond those set forth explicitly above are also possible, and will be immediately apparent to those of skill in the art in light of this disclosure. For example, particular components described for particular implementations can also be used for analogous purposes in other implementations, even where they are not explicitly described. For instance, a self complementarity score such as described as potentially being usable in a fitness function for the process of FIG. 10 could potentially be used as a sequence level fitness function in evolutionary processes such as shown in FIG. 13. Similarly, fitness functions such as described in the context of FIG. 13 could be used in implementations such as discussed in the context of FIG. 10. Similarly, the manufacturability evaluation function of FIGS. 12A-12B could be used make the determination shown in block 802 of whether a codon sequence satisfies a set of constraints in implementations following FIG. 8. As another example, in some cases, an evolutionary approach such as described in the context of FIG. 10, 13 or 18 could be used in a system which allowed a user to specify certain required codon subsequences, such as described in the context of block 807 of FIG. 8. In such a case, the evolutionary algorithm in question may accommodate the user specified subsequences by holding them constant from generation to generation (e.g., by assigning them a mutation probability of zero), thereby ensuring that the final optimized sequence would satisfy all constraints provided by the user. Other feature combinations are also possible, and will be immediately apparent to those of skill in the art in light of this disclosure. Accordingly, the protection provided by this document, or any related document, should not be treated as being limited to the examples and variations explicitly described, and instead should be treated as being defined by the claims in such document.

What is claimed is:

1. A method comprising:
receiving a target amino acid sequence;
generating a plurality of candidate codon sequences, wherein:
each candidate codon sequence codes for the target amino acid sequence;
the plurality of candidate codon sequences comprises a set of initial codon sequences and one or more sets of final codon sequences;
generating the plurality of candidate codon sequences comprises generating each of the one or more sets of final codon sequences based on the set of initial codon sequences; and
for each set of final codon sequences,
that set of final codon sequences corresponds to a vector from a set of vectors in a design space; and
each codon sequence in that set of final codon sequences is farther from an origin than any codon sequence from the set of initial codon sequences;
and
selecting, from one of the one or more sets of final codon sequences, an optimized codon sequence.

2. The method of claim 1, wherein:
generating each of the one or more sets of final codon sequences based on the set of initial codon sequences comprises, for each set of final codon sequences from the one or more sets of final codon sequences:
for each generation in a set of generations:
generating a set of new candidate codon sequences by creating a set of mutant codon sequences based on a set of previously generated candidate codon sequences;
for each candidate codon sequence in the set of new candidate codon sequences, calculating a fitness score for that candidate codon sequence using a fitness function corresponding to the vector corresponding to that set of final codon sequences; and
determining whether a termination condition is satisfied;
for each generation in the set of generations other than a final generation, wherein the termination condition is determined to be satisfied in the final generation:
identifying a set of candidate codon sequences, based on the identified set of candidate codon sequences not including any candidate codon sequence with a lower fitness score than any candidate codon sequence not comprised by the identified set of candidate codon sequences, as the set of previously generated candidate codon sequences to use for generating the new set of candidate codon sequences in a directly following generation from the set of generations;
identifying a set of previously generated candidate codon sequences which are farthest from the origin along the vector corresponding to that set of final codon sequences; and
generating a set of new candidate codon sequences by creating a set of mutant codon sequences based on the sequences from the identified set of previously generated candidate codon sequences;
and
after determining that the termination condition is satisfied, selecting candidate codon sequences for that set of final codon sequences.

3. The method of claim 2, wherein:
the set of initial codon sequences consists of a single codon sequence which codes for the target amino acid sequence;
the one or more sets of final codon sequences consists of a single set of final codon sequences;
the set of final codon sequence consists of a single candidate codon sequence; and
selecting the customized codon sequence is performed by designating the single candidate codon sequence from the single set of final codon sequences as the customized codon sequence.

4. The method of claim 2, wherein, for each generation in the set of generations, generating the set of new candidate codon sequences by creating the set of mutant codon sequences based on the set of previously generated candidate codon sequences comprises:
identifying a previously generated candidate codon sequence as a parent candidate codon sequence based on the parent candidate codon sequence having a fitness score which is not lower than the fitness score for any other previously generated candidate codon sequence;
selecting one or more positions in the parent codon sequence as mutation positions; and
defining a child candidate codon sequence by:
for each position in the parent codon sequence which is comprised by the mutation positions, defining the child candidate codon sequence as having the same codon in that position as the parent codon sequence;
for each position in the parent codon sequence which is comprised by the mutation positions, defining the child candidate codon sequence as having a codon in that position which is synonymous with the codon in that position in the parent codon sequence.

5. The method of claim 4, wherein, for each generation in the set of generations, selecting one or more positions in the parent codon sequence as mutation positions comprises:
at each position from the parent codon sequence, calculating a secondary structure at that position; and
selecting the mutation positions based on the calculated secondary structures.

6. The method of claim 5, wherein selecting mutation positions based on the calculated secondary structures is performed by randomly selecting mutation positions based on the calculated secondary structures.

7. The method of claim 1, wherein generating each of the one or more sets of final codon sequences based on the set of initial codon sequences comprises, for each set of final codon sequences from the one or more sets of final codon sequences:
for each step in a sequence of steps:
identifying a set of previously generated candidate codon sequences which are farthest from the origin along the along the vector corresponding to that set of final codon sequences;
generating a codon weighting table comprising weights based on codon frequencies from the identified set of previously generated candidate codon sequences; and
generating a set of new candidate codon sequences based on the codon weighting table;
and
after completing the sequence of steps, selecting candidate codon sequences for that set of final codon sequences.

8. The method of claim 7, wherein generating the set of new candidate codon sequences based on the codon weighting table comprises, for each of a set of potential candidate codon sequences:
performing a set of generation acts comprising:
adding a single codon to that potential candidate codon sequence at a probability from the codon weighting table, and at a closest unoccupied location to a first end of that potential candidate codon sequence; and
determining if that potential candidate codon sequence satisfies a set of constraints, wherein the set of constraints comprises a set of manufacturability constraints;
repeating the set of generation acts until a condition from a set of conditions is satisfied, wherein the set of conditions comprises:
that potential candidate codon sequence codes for the target amino acid sequence without violating the set of constraints; and
that potential candidate codon sequence is determined to not satisfy the set of constraints.

9. The method of claim 8, wherein for each of the set of potential candidate codon sequences, the first end of that potential candidate codon sequence is a 5′ end of that potential candidate codon sequence.

10. The method of claim 8, wherein:
generating the set of new candidate codon sequences based on the codon weighting table comprises:
for each of a first subset of the set of potential candidate codon sequences:
making a set of positive determinations for that potential candidate codon sequence, wherein the set of positive determinations comprises determining that that potential candidate codon sequence codes for the target amino acid sequence, and determining that that potential candidate codon sequence satisfies the set of constraints; and
based on making the set of positive determinations, adding that potential candidate codon sequence to the set of new candidate codon sequences;
for each of a second subset of the set of potential candidate codon sequences:
determining that that potential candidate codon sequence does not satisfy the set of constraints; and
based on determining that that potential candidate codon sequence does not satisfy the set of constraints, adding that potential candidate codon sequence to a failed subsequences table;
and
the set of constraints comprises not matching any sequences in the failed subsequences table.

11. The method of claim 8, wherein:
for each of the set of potential candidate codon sequences, the set of generation acts comprises checking if the closest unoccupied location to the first end of that potential candidate codon sequence corresponds to a fixed codon subsequence; and
for at least one of the set of potential candidate codon sequences, at least one repetition of the set of generation acts comprises, based on determining that the closest unoccupied location to the first end of that potential candidate codon sequence corresponds to the fixed codon subsequence, adding the fixed codon subsequence to that potential candidate codon sequence at the closest unoccupied location to the first end of that potential candidate codon sequence.

12. The method of claim 1, wherein selecting the customized codon sequence from one of the one or more sets of final codon sequences comprises:
for each codon sequence from the one of the one or more sets of final codon sequences, calculating a self-complementarity score for that final codon sequence by performing acts comprising:
generating a set of subsequences for that final codon sequence, wherein each subsequence from the set of subsequences has a length which is equal to the length of each other subsequence from the set of subsequences, and wherein the set of subsequences comprises:
each subsequence of that final codon sequence which has the length of each subsequence from the set of subsequences; and
each subsequence of a reverse complement of that final codon sequence which has the length of each subsequence from the set of subsequences;
for each subsequence from the set of subsequences for that final codon sequence, comparing that subsequence with each other subsequence from the set of subsequences, and creating a set of distance scores comprising one distance score for each of those comparisons; and
determining the self-complementarity score by combining the sets of distance scores for each of the subsequences from the set of subsequences;
and
selecting the customized codon sequence from the one of the one or more sets of final codon sequences based on the self-complementarity scores of the codon sequences from the one of the one or more sets of final codon sequences.

13. The method of claim 12, wherein:
for each codon sequence from the one of the one or more sets of final codon sequences:
the length of each subsequence from the set of subsequences is 22 nucleotides; and
for each comparison between two subsequences from the set of subsequences for that final codon sequence, creating the distance score for that comparison comprises executing instructions operable to:
assign distance scores which decrease as the number of differences between the compared subsequences increases, when the number of differences between the compared subsequences is greater than zero and less than a threshold difference level; and
assign a minimum distance score when the number of differences between the compared subsequences is greater than the threshold difference level;
and
selecting the customized codon sequence from the one of the one or more sets of final codon sequences comprises a final codon sequence with a minimum self-complementarity score.

14. The method of claim 1, wherein the design space has at least two dimensions, the at least two dimensions comprising a first dimension and a second dimension, wherein the first dimension and the second dimension are different, and each of the first dimension and the second dimension is selected from:
minimum free energy;
codon adaptation index;
summed frequencies of G and C nucleotides;
frequency of U nucleotides;
summed or localized probabilities of unpaired bases after folding;
modeled or estimated half-life;
windowed Trifonov linguistic complexity;
global Trifonov linguistic complexity;
windowed sequence entropy;
global sequence entropy;
windowed DUST complexity score;
global DUST complexity score; and
self-complementarity score, wherein, for each candidate codon sequence from the set of candidate codon sequences, a self-complementarity score is calculated for that candidate codon sequence by performing acts comprising:
generating a set of subsequences for that codon sequence, wherein each subsequence from the set of subsequences has a length which is equal to the length of each other subsequence from the set of subsequences, and wherein the set of subsequences comprises:
each subsequence from the set of subsequences; and
each subsequence of a reverse complement of that codon sequence which has the length of each subsequence from the set of subsequences;
for each subsequence from the set of subsequences for that codon sequence, comparing that subsequence with each other subsequence from the set of subsequences, and creating a set of distance scores comprising one distance score for each of those comparisons; and
determining the self-complementarity score by combining the sets of distance scores for each of the subsequences from the set of subsequences.

15. The method of claim 1, wherein the method comprises:
receiving a set of one or more untranslated region sequences; and
generating the plurality of candidate codon sequences comprises, for each candidate codon sequence, applying a validation function to that candidate codon sequence by applying the validation function to a nucleotide sequence which comprises that candidate codon sequence.

16. The method of claim 1, wherein:
the method comprises generating a seed codon sequence based on providing the target amino acid sequence to a program configured to:
generate a plurality of codon sequences which code for the target amino acid sequence; and
identify an output codon sequence which has a distance from an origin in a design space corresponding to that program which is greater than an average distance from the origin in the design space corresponding to that program for all of the plurality of codon sequences generated by that program;
the seed codon sequence is the output codon sequence identified by the program; and
the set of initial codon sequences comprises the seed codon sequence.

17. The method of claim 16, wherein the program configured to identify the output codon sequence is configured to generate the plurality of codon sequences which code for the target amino acid sequence in executing a search algorithm.

18. The method of claim 1, wherein:
generating each of the one or more sets of final codon sequences based on the set of initial codon sequences comprises, for each set of final codon sequences from the one or more sets of final codon sequences:
for each generation in a set of generations:
generating a set of new candidate codon sequences based on creating a set of mutant codon sequences based on a set of previously generated candidate codon sequences; and
determining whether a termination condition is satisfied.

19. A non-transitory computer readable medium having stored thereon instructions operable to, when executed, cause a computer to perform the method of claim 1.

20. A system comprising a computer programmed to perform the method of claim 1.
